(19) United States
(12) Patent Application Publication
(10) Pub. No.: US 2026/0367079 A1
(43) Pub. Date: Nov. 19, 2026

(54) IDENTIFICATION AND/OR ISOLATION OF T CELL-COMPRISING CELL-CELL COMPLEXES AND USE THEREOF

(71) Applicant: STICHTING HET NEDERLANDS KANKER INSTITUUT-ANTONI VAN LEEUWENHOEK ZIEKENHUIS, AMSTERDAM (NL)

(72) Inventors: Daniel Simon PEEPER, AMSTERDAM (NL); Sofía IBÁÑEZ MOLERO, AMSTERDAM (NL); Johanna VELDMAN, AMSTERDAM (NL)

(21) Appl. No.: 19/136,532

(22) PCT Filed: Dec. 8, 2023
(86) PCT No.: PCT/NL2023/050645

§ 371 Date: Jun. 6, 2025

(30) Foreign Application Priority Data
Dec. 9, 2022 (NL) 2033706

(51) Int. Cl. G01N 33/569; C12Q 1/6876; G01N 1/30

(52) U.S. Cl. CPC G01N 33/56972; C12Q 1/6876; G01N 1/30; C12Q 2600/158; G01N 2333/7051; G01N 2570/00

(57) ABSTRACT

The current invention relates to the identification of specific T cells, antigen-presenting cells (APC) and/or tumor cells in T cell clusters identified in a sample obtained from a subject having cancer or being suspected of having cancer. The T cells and/or APC identified with the current invention are useful in the treatment of cancer, in particular in the subject from which the sample was obtained, for example in adoptive T cell therapy (ACT), for example but not limited to TIL therapy. The TCR from the T cells identified with the current invention may be used in preparing engineered T cells that are likewise useful in the treatment of cancer.

### FIELD OF THE INVENTION

This invention pertains in general to the isolation of cells from a subject. This invention pertains to the identification and isolation of cells from T cell comprising cell-cell complexes obtained from a sample of a subject. The cells thus obtained are useful in treatment of cancer, in particular by immunotherapy.

In particular, the current invention relates to the identification of specific T cells, antigen-presenting cells (APC) and/or tumor cells from a sample obtained from a subject having cancer or being suspected of having cancer. The T cells and/or APC identified with the current invention are useful in the treatment of cancer, in particular in the subject from which the sample was obtained. In particular the T cells are useful in adoptive T cell therapy (ACT), for example but not limited to TIL therapy. The T cell receptor (TCR) from the T cells identified with the current invention may be used in preparing engineered T cells that are likewise useful in the treatment of cancer.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Immunotherapies have revolutionized cancer treatment in recent years, improving significantly the clinical outcome of patients. From immune check points to cell therapies, the majority of immunotherapies aim to unlock and exploit the cytotoxic T cell potential to identify tumor cells and eradicate them. While several immune cell types contribute to therapy-induced antitumor immunity, the process typically converges at the step whereby highly specific recognition of tumor cells by cytotoxic CD8 T cells results in tumor elimination.

When this immune antitumor response somehow fails, it commonly leads to therapy resistance. Indeed, despite the clinical success of immunotherapies in cancer, there are still many patients failing to durably respond, owing to resistance mechanisms (Kalbasi & Ribas, 2020; Sharma et al., 2017) 2 Amongst many other mechanisms, the lack of (neo)antigens or deficiencies in the antigen presentation machinery prevent tumor recognition by T cells.

In the last two decades, several types of immunotherapies have been developed. Indeed, there are several international efforts, both in the academic (Arnaud et al., 2022; Kato et al., 2018; Lu et al., 2021) and commercial pharma space (Neogene and many others), which, next to immune checkpoint blockade, ICB, aim to identify tumor-associated antigens (TAA)/TCR matches for adoptive TCR T cell therapy.

The knowledge that tumor antigen-specific T cells are pivotal for the control of cancer is being therapeutically harnessed also by other clinical strategies such as adoptive T cell therapy (ACT), for example but not limited to TIL therapy (Rapoport et al., 2015; Rosenberg et al., 1986). TIL adoptive therapy is based on ex vivo expanded TILs, for example in particular in melanoma (Van Den Berg et al., 2020). TILs are produced from cell suspensions prepared from freshly resected tumor material. Cultured TILs are expanded to remarkably high numbers in a so-called rapid expansion protocol (REP) for 2 weeks, for subsequent patient infusion.

TCR/TAA strategies are based on targeted genomic sequencing of cancer patient biopsies, to genetically analyze both tumor cells and T cells for neoantigen and TCR discovery, respectively. Ultimate goal is to develop individualized T cell therapies, whereby multiple T cell clones with predicted antitumor activity are admixed and infused back into the patient. It is, however, a method that is time-, labor-, and cost-intensive. Moreover, it relies fully on the bioinformatic prediction of TAAs, which is imperfect to date, and on large and complex screening methods. Similar to TIL therapy, this approach does not consider any functional parameter of the tumor:T cell interaction nor any validation on the patients' tumors.

In light of this, new products, compositions, methods and uses for in the treatment of cancer would be highly desirable but are not yet readily available. In particular, there is a clear need in the art for cheaper, faster, reliable, efficient, and reproducible products, compositions, methods and uses that allow to be used in the treatment of cancer, in particular that better allow to identify and isolate tumor-reactive T cells or other useful cells from patients, and that may be used in such cancer treatment. Accordingly, the technical problem underlying the present invention can been seen in the provision of such products, compositions, methods and uses for complying with any of the aforementioned needs, or at least providing the public with a useful choice. The technical problem is solved by the embodiments characterized in the claims and herein below.

### SUMMARY OF THE INVENTION

As embodied and broadly described herein, the present invention is directed to the surprising finding that from a sample obtained from a subject, preferably a subject having cancer or having a tumor, T cell-comprising cell-cell complexes may be obtained and that the T cells comprised in such T cell-comprising cell-cell complexes are in particular suitable in the treatment of cancer. In addition, APC and tumor cells may be identified and obtained from such T cell comprising cell-cell complexes, and may likewise be used in, for example the treatment of cancer and/or in cancer research. The T cell-comprising cell-cell complexes may be identified in both fresh material obtained from the subject or from material that has been frozen.

Therefore, in an aspect, the invention provides for a method of enrichment of T cells, particular enrichment of T cells that are present in a tumor in the subject or that are present in the blood, or in pleural effusions, and that are tumor-reactive.

With the invention, the number of bystander T cells (i.e., T cells that, although being present in tumor tissue, are not, or only to a limited extent, reactive towards tumor cells) is reduced. This enrichment of tumor-reactive T cells thus allows for obtaining an improved population of expanded tumor-reactive T cells (as part of, for example adoptive T cell therapy (ACT), for example but not limited to TIL therapy), by excluding such bystander T cell and enriching for tumor-reactive T cells prior to expanding the T cell population.

The invention also allows for the isolation of other cells that are comprised in the T cell-comprising cell-cell complexes, including APCs and/or tumor cells that are conjugated to the T cell(s) in the T cell comprising cell-cell complex.

The invention also allows for the identification of a TCR that is expressed by a T cell that is expressed or comprised in a T cell comprising cell-cell complex. The identification of such TCR is useful, for example, in providing engineered T cells based on such TCR, or comprising the tumor antigen-recognizing part of such TCR. This could be used, for example, in providing a therapeutic product comprising a collection of engineered T cells with, for example 1-10 or 5-10 different TCRs targeting distinct TAAs./pct

The invention thus allows for the enrichment of TAA-reactive killer T cells.

The invention also allows for the identification and/or isolation of T cell-comprising cell-cell complexes from a blood sample obtained from the subject, for example T cell-tumor cell complexes and/or T cell-APC complexes (or clusters), as well as the cell in these complexes.

The invention also allows for the identification and/or isolation of T cell-comprising cell-cell complexes from a lymph node sample obtained from the subject, for example T cell-tumor cell complexes and/or T cell-APC complexes, as well as the cells in these complexes.

The invention also allows for the identification and/or isolation of the T cell-comprising cell-cell complexes from a tissue sample, tumor tissue sample, a blood sample and/or a lymph node sample obtained from a subject, preferably a subject having a tumor.

The protocol is based on all T cells obtained from the resected tumor material. The invention overcomes the problem of the absence of enrichment and/or functional validation for tumor reactivity of the T cells obtained from a subject using the method described in the prior art, or at least provides for an alternative way to obtain such T cells.

### BRIEF DESCRIPTION OF THE DRAWINGS

Embodiments of the invention are further described hereinafter with reference to the accompanying drawings, in which:

FIG. 1: A: Representative scheme of in vitro matched T cell:tumor model. B:Measurement of percentage doublets (flow cytometry measurement of % mPlum+CD8-Pacific Blue+ cells within the live cell population) in a co-culture of a MART-1 expressing melanoma cell line together with MART-1 TCR expressing CD8 T cells. C: A cancer cell line panel was analyzed for doublet formation, as described in B with a mix of T cells expressing MART-1 TCR or not (untransduced). D: Measurement of CD69 cell surface expression by flow cytometry in untransduced or MART-1-specific T cells upon co-culture with MART-1 expressing D10 melanoma cell line at the indicated time points. Visualized as percentage of CD69+ cells within the doublet population. E: Imagestream® analysis of cell:cell doublets and larger cell clusters from a co-culture of MART-1-expressing A875 melanoma cells and MART-1 TCR-expressing T cells. Tumor cells expressed mPlum and T cells were labelled with Cell Trace Violet. Cells were co-cultured for 4 h at a 1:1 (tumor: T cell) ratio. F: Measurement of doublet formation (left) and doublet activation by CD69 expression (right) in MART-1 expressing D10 co-culture with MART-1 high affinity TCR-expressing T cells, MART-1 low affinity TCR-expressing T cells or untransduced T cells. PMA/lonomycin (ION) was used as a positive control for activation. G: Flow cytometry analysis of a 40:60 mixture of MART-1 TCR T cells and untransduced T cells in a co-culture with the MART-1-expressing cancer cell line panel. “T cells only” indicates the T cell sample before the co-culture. For each individual cell line, MART-1-specific T cells were measured in singlets and doublets. Cells were incubated at 2:1 (T cell:tumor) ratio for 4 h. Mouse TCR beta is used as measure of the MART-1-specific TCR.

FIG. 2: A: Imagestream® analysis of doublets (top) and triplets/clusters (bottom) from a melanoma patient sample. Different channels used for indicated cell type (Tumor, APC, T cell or merge). B: Imagestream® analysis as described in A from patient's blood sample. CTC indicates circulating tumor cells.

FIG. 3: A-D: direct analysis of clinical samples before REP protocol. A: ImageStream measurement of percentage tumor (NGFR-PE+/CD146-PE+):T cell (CD8-APC+) doublets in a melanoma patient sample, measured as percentage of live cells. B: Overview of percentage of T cell:tumor cell doublets, T cell:APC doublets and T cell singlets in five melanoma patient samples, measured as percentage of live cells. C:Measurement of percentage T cell:tumor cell doublets or larger clusters in a patient sample, processed either with or without pan-caspase inhibitor (Z-VAD-FMK). D: Fold-change increase of median fluorescent intensity (MFI) CD69 (activation marker) cell surface expression in T cell:tumor doublets and T cell:APC doublets compared to T cell singlets in one patient sample. E: Possible workflow of the REP protocol. In short, tumor digest was stained, T cell:tumor doublets, T cell:APC doublets and T cell singlets were sorted and expanded with a REP. After expansion, T cells were rested for 3 days after which several functional assays were performed (see F-J). In parallel, tumor cells were sorted, and, when possible, a tumor cell line was established. Alternatively, tumor digest is used for functional assays. F-J: Secondary co-cultures of patient tumor and T cells after the REP protocol. The Figures contain data of one REP experiment, each condition with three technical replicates. F: Percentage of T cell:tumor cell doublets after a 4 h secondary co-culture of CTV-stained REP-ed T cells together with the cell line established from the patient's tumor. G: Tumor cell annexin V MFI (median fluorescence intensity; left) and percentage of tumor annexin V+ cells (right) in a 4 h secondary co-culture of CTV-stained REP-ed T cells together with the cell line established from the patient's tumor. Each co-culture was performed separately for the various input single or doublet cells. The tumor only sample was cultured without T cells. H: T cell activation, as measured by percentage of IFNy+, TNFa+ or IFNy+TNFa+ CD8+ T cells, after a 4 h secondary co-culture of CTV-stained REP-ed T cells with the patient's tumor cell line. I: Percentage of viable tumor cells relative to control (cultured without T cells) after a 7-day secondary co-culture with REP-ed T cells measured by cell titer blue. T cells were challenged twice, on day 0 in ratios 1:2, 1:1 and 2:1 (T cell:tumor cell) and on day 3 on which T cells were washed away and 25.000, 50.000 and 100.000 T cells were added to each well, respectively. J: Crystal violet staining of the experiment described in I. Tumor cells were seeded at two different cell concentrations on day 0, 10.000 cells/well or 25.000 cells/well.

FIG. 4. Antigen-presenting cell (APC) phenotypes identified in melanoma tumors. Different frequencies of APC phenotypes in APCs from clusters versus APC singlets. Higher bars indicate a higher frequency of that phenotype in APCs from clusters. Each dot represents an individual patient.

FIG. 5. Functionality of expanded T cells derived from T cell:tumor doublets/clusters and T cell:APC doublets/clusters compared to T cell singlets in non-small cell lung cancer and penile cancer patients. A) Quantification of TNF, IFNy production and CD137 upregulation by expanded CD8+ T cells derived from singlets (blue-first column in all sets of bars), tumor cell clusters (purple-second column in all sets of bars) or APC clusters (green-third column in all sets of bars) after a 4 h or 24 h co-culture with autologous non-small cell lung (NSCLC) tumor digest. Statistical analysis was performed using an unpaired t-test. Significance is indicated in comparison to T cells from singlets. Two patients are shown, indicated by numbers. B) Quantification of CD137 upregulation by expanded CD8+ T cells derived from singlets (blue-first column in the sets of bars) and APC clusters (green-third column in the set of bars) after a 24 h co-culture with autologous penile tumor digest. There were not enough T cells from tumor clusters sorted and expanded to perform this experiment (indicated with a cross). Statistical analysis was performed using an unpaired t-test. * P<0.05; ** P<0.01; *** P<0.001; **** P<0.0001. data in this figure derive from clusters with tumor digest.

FIG. 6. Antigen-specific CD8+ T cells outcompete non-specific T cells in heterotypic tumor cell clusters. a, In vitro co-culture of FM6 human melanoma cells (mPlum+) with CD8+ T cells (CTV-stained) for 4 h and analyzed by flow cytometry. The plot shows heterotypic clusters, as judged by mPlum/CTV double-positive cells.b, In vitro co-culture of D10 human melanoma cells (mPlum+) with CD8+ T cells (CTV-stained) for 4 h and visualized with imaging flow cytometry (ImageStream®). Cells were additionally stained for HLA-A*02. c, Proportion of clustered CD8+ T cells from the total T cell population, after a 4 h in vitro co-culture of different cancer cell lines. One representative donor is shown (two additional donors were analyzed too) (n=3). Mean±S.D. Each data point represents a technical replicate. d, Schematic representation of in vitro competition assay in which tumor cells expressing MART-1 antigen were co-cultured with a mixture of CD8+ T cells containing a MART-1-specific or non-specific TCR and subsequently analyzed by flow cytometry. The final analysis evaluates the distribution of specific and non-specific CD8+ T cells in clusters and singlets. e, A875 human melanoma cells co-cultured with a 40:60 mix of MART-1-specific:non-specific T cells (Input). After 4 h, the percentage of MART-1-specific and non-specific T cells in clusters (C) and singlets(S) was assessed by flow cytometry. Average fold-change (Avg. FC) of MART-1-specific T cells in clusters over singlets was calculated. Mean±S.D. Statistical analysis was performed with a paired t-test. f, In vitro co-culture of A875 human melanoma cells with different mixtures of MART-1-specific:non-specific T cells. After 4 h, the percentage of MART-1-specific T cells in clusters versus singlets and Avg. FC was determined as described in (e). One representative donor is shown (two additional donors in Extended data FIG. 2b) (n=3). Mean±S.D. Statistical analysis was performed with a paired t-test. g, In vitro co-culture of different human cancer cell lines with a 5:95 mixture of MART-1-specific:non-specific T cells. After 4 h, the percentage MART-1-specific T cells in clusters versus singlets and Avg. FC was determined as described in (e). One representative donor is shown (two additional donors in Extended data FIG. 2d) (n=3). Mean±S.D. Statistical analysis was performed with a paired t-test. * P<0.05; ** P<0.01; *** P<0.001.

FIG. 7. Heterotypic clusters of CD8+ T cells with tumor and/or APCs from clinical melanoma specimens. a, Schematic representation of tumor sample collection and processing. Fresh tumor samples were obtained, cut into small pieces and briefly enzymatically digested to obtain a cell suspension and analyzed by flow cytometry. b, Representative flow cytometry plot of a melanoma tumor digest from patient 1 obtained as described in (a). The tumor digest was stained for tumor cell markers CD146 and NGFR, T cell marker CD8 and APC marker CD11c. Clusters were identified as double-positive cell populations. c, Proportion T cell:Tumor (Tum), T cell:APC and T cell:Tumor:APC clusters from the total CD8+ T cell population for all ten melanoma patients analyzed. Mean±S.E.M. Each colored data point represents an individual patient (n=10). d, Correlation between percentage CD8+ T cell infiltration and percentage T cell:Tumor (Tum) (left) or T cell:APC (right) clusters. Statistical analysis was done using Pearson correlation coefficients. Each data point represents an individual patient (n=10).e, Tumor digest of patient 1 in (b) visualized by imaging flow cytometry (ImageStream®). Representative single cells (top) and clusters with different compositions (bottom) are shown.

FIG. 8. Frequency of cell states in patient 8 visualized for CD8+ T cells from either singlets, tumor cell clusters or APC clusters. One representative patient is shown (three additional patients were analyzed but data are not shown) (n=4). Statistical analysis was performed with a false-discovery rate (FDR)-adjusted Fisher's exact test. A significant increase in population frequency of clustered T cells versus singlet T cells is indicated.

FIG. 9. Expanded CD8+ T cells from clusters show increased cytokine production and killing of autologous tumor cells. a, Schematic representation of workflow for T cell activation and tumor killing assays. From tumor digests, CD8+ T cell:tumor cell clusters, CD8+ T cell:APC clusters, and T cell singlets and tumor cell singlets were sorted. Sorted T cells from all populations were subsequently expanded using a rapid expansion protocol (REP). After a resting period, T cells were co-cultured with ex vivo-expanded autologous tumor cells. b, Representative flow cytometry plots showing TNF and IFNy production by expanded CD8+ T cells derived from either singlets (left, blue), tumor cell clusters (middle, purple) or APC clusters (right, green) after a 4 h co-culture with autologous tumor cells (patient 2). c, Standardized score (Z-score) for production of TNF and IFNy by expanded CD8+ T cells derived from either singlets, tumor cell clusters or APC clusters after a 4 h co-culture with autologous tumor cells (n=3). Standardization was done per cytokine and per patient. For each patient the mean of three technical replicates is shown. Statistical analysis was performed on the non-normalized data using an unpaired t-test. Significance is indicated in comparison to T cells from singlets. d, Tumor cell killing capacity of expanded CD8+ T cells derived from either singlets, tumor cell clusters or APC clusters against autologous melanoma cells, as determined by a CellTiter-Blue assay (n=6). Percentage tumor cell killing was determined in comparison to untreated tumor cells. Statistical analysis was performed using a 2-way ANOVA, followed by a Dunn's multiple comparisons test. Significant increases in killing by clustered CD8+ T cells compared to T cells from singlets is indicated. Average fold-change (Avg. FC) killing of T cells from clusters over T cells from singlets is indicated (top). Mean±S.D. Each data point represents a technical replicate. * P<0.05; ** P<0.01; *** P<0.001; **** P<0.0001

FIG. 10. T cells in cell:cell complexes from blood (a) and pleural effusions (b). Cytotoxic T cells (CTC) and APCs in clusters with T cells (CD4 and CD8) in blood (top) and pleural effusion samples (bottom) were visualized by Imagestream®, following the previously disclosed methodology.

### DESCRIPTION

### Definitions

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a cell” includes a combination of two or more cells, and the like. For example, a method for administrating a T cell includes the administrating of a plurality of T cells (e.g., 10's, 100's, 1000's, 10's of thousands, 100's of thousands, millions, or more).

As used herein, “about” and “approximately”, when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed invention. Unless otherwise clear from context, all numerical values provided herein include numerical values modified by the term “about.”

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . −10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps, or components. It also encompasses the more limiting “to consist of.”

As used herein, “conventional techniques” or “methods known to the skilled person” refer to a situation wherein the methods of conducting the conventional techniques used in methods of the invention will be evident to the skilled worker. The practice of conventional techniques in molecular biology, biochemistry, cell culture, genomics, sequencing, medical treatment, pharmacology, immunology, and related fields are well-known to those of skill in the art and are discussed, in various handbooks and literature references.

As used herein, “exemplary” or “for example” means “serving as an example, instance, or illustration,” and should not be construed as excluding other configurations, including those disclosed herein.

As used herein, “adoptive T cell therapy (ACT)” means therapy wherein involving transfer of T-cells into a subject. The cells may have originated from the patient or from another individual. ACT is also called T cell therapy, adoptive immunotherapy, and immune cell therapy. ACT includes, but is not limited to tumor-infiltrating lymphocytes (TIL) therapy, engineered T Cell Receptor (TCR) therapy, Natural Killer (NK) Cell Therapy, and CAR-T cell therapy.

As used herein, an “antigen presenting cells” or “APCs” refers to a heterologous group of immune cells that can process and present antigens for stimulating responses of certain lymphocytes (e.g., T cells and B cells). Classical APCs include, for example, dendritic cells, macrophages, B cells, and neutrophils.

As used herein, an “apoptosis inhibitor” is a substance (e.g., a molecule, biomolecule, small organic compound) that inhibits one or more processes in an apoptosis pathway, so that apoptosis of a cell or cells is reduced or eliminated. Apoptosis can be quantified in a number of ways, including, without limitation, treating cells with propidium iodide and annexin-V FITC, with staining quantified by FACS analysis. Other methods include, without limitation, a TUNEL assay and DNA fragmentation. Using these assays, the number or proportion of cells undergoing apoptosis can be determined.

As used herein, “cancer” refers to the physiological condition in mammals that is typically characterized by unregulated cell growth. The terms “cancer,” “neoplasm,” and “tumor,” are often used interchangeably to describe cells that have undergone a malignant transformation that makes them pathological to the host organism. Primary cancer cells can be distinguished from non-cancerous cells by techniques known to the skilled person. A cancer cell, as used herein, includes not only primary cancer cells, but also cancer cells derived from such primary cancer cell, including metastasized (secondary) cancer cells, and cell lines derived from cancer cells. Examples include solid tumors and non-solid tumors or blood tumors. Examples of cancers include, without limitation, leukemia, lymphoma, sarcomas, and carcinomas (e.g., colon cancer, pancreatic cancer, breast cancer, ovarian cancer, prostate cancer, lung cancer, melanoma, lymphoma, non-Hodgkin lymphoma, colon cancer, (malignant) melanoma, thyroid cancer, papillary thyroid carcinoma, lung cancer, non-small cell lung carcinoma, and adenocarcinoma of lung.). Treatment of a cancer in a subject includes the treatment of a tumor in the subject.

As used herein, a “cell death inhibitor” refers to means and/or compounds that are able to inhibit cell death triggered by T cells. Although not limited thereto, examples of such “cell death inhibitors” include apoptosis inhibitors and/or caspase inhibitors. Example of such caspase inhibitors include, but are not limited to ZVAD (zVAD-fmk), VAD, YVAD, ZYVAD-fmk, XIAP, BIR, clAP-1, clAP-2, Quinoline-Val-Asp-Ch2-O-Ph (QVD), or BAF

As used herein, “in vivo” refers to an event that takes place in a subject's body; “in vitro” refers to an event that takes places outside of a subject's body. For example, an in vitro assay or method encompasses any assay or method conducted outside of a subject. In vitro assays or methods encompass cell-based assays in which cells, alive or dead, are employed. In vitro assays also encompass a cell-free assay in which no intact cells are employed.

As used herein, the term “marker” is used to describe the characteristics and/or phenotype of a cell. Markers can be used for selection of cells comprising characteristics of interests. Markers will vary with specific cells. Markers are characteristics, whether morphological, functional, or biochemical characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids, and steroids. Examples of morphological markers include shape, size, and nuclear to cytoplasmic ratio. Examples of functional markers include the ability to migrate under particular conditions and the ability to differentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

As used herein, “sample” when referring to a tumor or any other biological material referenced herein, means a sample that has been removed from the subject; thus, none of the testing methods described herein are performed in or on the subject.

As used herein, a “T cell” can be selected from the group consisting of inflammatory T-lymphocytes, cytotoxic T-lymphocytes, regulatory T-lymphocytes, or helper T-lymphocytes. In another embodiment, said cell can be derived from the group consisting of CD4+ T-lymphocytes and CD8+ T-lymphocytes. The T cell may be a alpha-beta T cells. The T cell may be a gamma-delta T cell. The T cell may be a Natural Killer T cell. The T cell may be an engineered T cell, for example CAR T cell that was provided to the subject prior to performing the method of the invention. They can be extracted from blood, from tissue and/or from tumor tissues. In another embodiment, said T cell is part of a mixed population of cells which present different phenotypic characteristics.

As used herein, “treatment”, “treating”, “palliating”, “alleviating” and “ameliorating” in the context of a subject to be treated, all refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, “tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors.

### DETAILED DESCRIPTION

The invention is defined herein, and in particular in the accompanying claims. Subject-matter which is not encompassed by the scope of the claims does not form part of the present claimed invention.

It is contemplated that any method, use, or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

Any references in the description to methods of treatment refer to the compounds, pharmaceutical compositions, and medicaments of the present invention for use in a method for treatment of the human (or animal) body by therapy.

As embodied and broadly described herein, the present invention is directed to the surprising finding that in a sample obtained from a (human) subject, T cell-comprising cell-cell complexes can be identified and/or isolated. The T cells in the T cell-comprising cell-cell complexes are believed to be conjugated to at least one other cell in the T cell-comprising cell-cell complexes via TCR-antigen binding. Surprisingly, such T cell-comprising cell-cell complexes are sufficiently stable that they can be identified and isolated from a sample (e.g., tissue or blood, comprising cells) obtained from a subject despite experimental stress and despite the fact the cells may be obtained from tumor tissue normally characterized by a hostile tumor microenvironment. More surprisingly, the T cells thus obtained can be expanded and be re-introduced in a subject, in particular the subject from which the sample was obtained. In addition, the TCR of the T cells identified and/or isolated with the method of the invention may be identified, for example, may be sequenced (amino acids and/or DNA/RNA sequence) and use to provide for engineered T cells that are useful in the treatment of cancer.

In addition, the invention allows for the identification and/or isolation of cells that interact with the T cells that are comprised in the T cell comprising cell-cell complex, for example cells that are conjugated with the T cell comprised in the cell-cell complex via TCR-antigen binding, such a tumor cells and/or antigen-presenting cells expressing an antigen on the cell surface. These cells are useful in, for example, establishing a patient-derived material, e.g., a patient-derived tumor cell line or a patient-derived xenograft (PDX). At the same time these cells, in particular certain APCs, in particular wherein the APC is an APC natural or endogenous to the subject or a non-malignant APC, may be useful in the treatment of cancer as well, in particular in the subject from which the sample was obtained.

This current invention has no reliance on computational neoantigen prediction, and no library generation and screening are necessary. The invention relies only on T cells in conjugation with other cells, including tumor cells and APCs such as dendritic cells (DCs), or both, and conjugation can be used as a functional key parameter for antitumor T cells.

Therefore, in some embodiments, the invention provides for a method for isolating T cells from a sample obtained from a subject, the method comprising at least the steps of:
(i) obtaining or providing a sample from a subject, wherein the sample is
   (a) a tissue sample, for example a lymph node sample; and/or
   (b) a blood sample,
(ii) treating the sample by:
   (a) in case of a tissue sample, for example a lymph node sample, dissociating the tissue sample; and/or
   (b) in case of a blood sample, lysing or removing erythrocytes from the blood sample, and
(iii) identifying in and/or isolating from the sample obtained after step (ii) cell-cell complexes comprising a T cell, and/or a T cell comprised in the cell-cell complex, and/or any other cell that is comprised in the cell-cell complex.

In some embodiments, the sample from the subject is a sample that comprises tumor cells. In some embodiment the sample that is obtained from the subject is a biopsy. In some embodiments the sample that is obtained from the subject is a patient-derived tumor fragment. In some embodiments the sample is a tumor digest. In some embodiments the sample is a lymph node or a lymph node sample. In some embodiments the sample taken from the subject is a pleural effusion sample. In some embodiment the sample from the subject is a blood sample or a sample obtained from the blood of a patient. In some embodiments the sample obtained from the subject may be frozen. In some embodiments, to the sample that is obtained from the subject, a cell death inhibitor, for example an apoptosis inhibitor, is added, preferably wherein the cell death inhibitor, for example an apoptosis inhibitor is added prior to freezing the sample. In some embodiments, to the sample that is obtained from the subject, a cell death inhibitor, for example an apoptosis inhibitor is added when thawing the sample. These and other aspects of this embodiment of the invention are further detailed herein.

Therefore, in a preferred embodiment of the invention, there is provided for a method for isolating T cells from a sample obtained from a subject, wherein the subject is diagnosed with cancer and/or is suspected of having cancer, the method comprising at least the steps of:
(i) obtaining or providing a sample from the subject, wherein the sample is
   (a) a tumor tissue sample; and/or
   (b) a blood sample, and/or
   (c) a lymph node sample
(ii) treating the sample by:
   (a) in case of a tumor tissue and/or lymph node sample, dissociating the tumor tissue and/or lymph node sample; and/or
   (b) in case of a blood sample, lysing or removing erythrocytes from the blood sample, and
(iii) identifying in and/or isolating from the sample obtained after step (ii) cell-cell complexes comprising a T cell, and/or a T cell comprised in the cell-cell complex.

The skilled person is well-aware of methods for obtaining a sample from a subject, including where the sample is a tumor tissue sample and/or a blood sample and/or a lymph node sample.

The skilled understands that the sample obtained from the subject must comprise cells.

The subject may be an animal of a human subject. The subject may be a subject that has been diagnosed with having cancer, but the subject may also be suspected of having cancer, although the diagnosis has either not been made or not been confirmed.

The current invention is not in particular limited to a specific type of cancer. The cancer may, for example, be selected from leukemia, lymphoma, sarcomas, and carcinomas (e.g., colon cancer, pancreatic cancer, breast cancer, ovarian cancer, prostate cancer, lung cancer, melanoma, lymphoma, non-Hodgkin lymphoma, colon cancer, (malignant) melanoma, thyroid cancer, papillary thyroid carcinoma, brain cancer, lung cancer, non-small cell lung carcinoma, and adenocarcinoma of lung).

Where, in the current invention, a sample is provided from a tumor tissue sample, the tumor is preferably a solid tumor. The tumor may be a primary tumor or a secondary tumor. The tumor may be a metastatic tumor.

Where, in the current invention, a sample is provided that is a blood sample, the blood sample may be obtained from the subject using methods well-known to the skilled person.

Where, in the current invention, a sample is provided from a lymph node sample, the lymph node sample may be obtained from the subject using methods well-known to the skilled person.

The samples used in the current invention may, prior to performing additional steps in the method of the invention be stored, for example, by freezing (using methods commonly used for freezing samples that comprise living cells). In addition, the sample may, prior to performing additional step in the method of the invention, be contacted with various materials including buffers and the like, as long at least part of the cells present in the sample obtained from the subject remain alive. The skilled person knows how to treat and/or manage such samples obtained from the subject.

In some embodiment of the invention, the sample obtained from the subject was already obtained before performing the steps of the method as disclosed herein. In such embodiment step (i) of the method of the invention relates to obtaining or providing a sample that was already obtained from the subject. In such embodiment step (i) relates to providing a sample that was obtained from the subject.

In a next step (ii) of the method of the invention, the sample is treated.

In the case of a tumor tissue sample (i.e., a sample obtained from a tumor that is present in the subject) and/or a lymph node sample, the tissue is treated such that the cells in the tissue are dissociated from the tumor tissue sample and/or the lymph node sample. Within the context of the invention, the tumor tissue needs to be processed to dissociate the cells from the tissue, such that the cells are no longer associated with the original tissue.

There are three primary methods for tissue dissociation and frequently the methods are combined. These include enzymatic dissociation, chemical dissociation, and mechanical dissociation. Enzymatic dissociation is the process of using enzymes to digest cut-up tissue pieces thereby releasing cells from tissue. Many distinct types of enzymes are used in this process, and they can also be used in combination, as is well-known to the skilled person. Chemical dissociation takes advantage of the fact that cations participate in the maintenance of intracellular bonds and the intracellular matrix. By introducing EDTA or EGTA, which binds these cations, the intercellular bonds are disrupted. Mechanical dissociation requires cutting, scraping, or scratching the tissue into small pieces, then the minced-up tissue is washed in medium in order to separate the cells from the tissue and sometimes gentle agitation is also used to help loosen the cells. Within the context of the current invention any suitable type of dissociation may be used. However, preferably dissociation is by enzymatic dissociation, using methods well-known to the skilled person, including those disclosed herein. Within the context of the current invention, dissociating the tumor tissue and/or lymph node sample is aimed at/achieves obtaining cell-cell clusters in suspension, including the T cell-comprising cell-cell complexes according to the invention. The skilled person, based on the current disclosure, understands how to obtain such cell-cell clusters in suspension.

In the case of a blood sample, the blood sample is treated to remove erythrocytes from the blood sample. The skilled person is well-aware of methods for removing or lysing erythrocytes from the blood sample in a way that other cells are not affected. For example, Red Blood Cell (RBC) Lysis Buffer is formulated for optimal lysis of erythrocytes in single-cell suspensions. This buffer normally contains ammonium chloride, which lyses red cells with minimal effect on lymphocytes when used as instructed, thus allowing the selective lysis of the erythrocytes, while leaving the other cell intact. Another example for removing the erythrocytes includes isolation with Ficoll-Plaque. Ficoll is a neutral, highly branched, high-mass, hydrophilic polysaccharide which dissolves readily in aqueous solutions. Ficoll-Paque is normally placed at the bottom of a conical tube, and blood is then slowly layered above Ficoll-Paque. The following layers will be visible after centrifugation in the conical tube, from top to bottom: plasma and other constituents, a layer of mono-nuclear cells called buffy coat (PBMC/MNC), Ficoll-Paque, and erythrocytes and granulocytes. This separation allows easy harvest of PBMCs. Within the context of the current invention, treatment of the blood sample is aimed at/achieves obtaining cell-cell clusters in suspension, including the T cell-comprising cell-cell complexes according to the invention. The skilled person, based on the current disclosure, understand how to obtain such cell-cell clusters in suspension.

In the method of the invention, cell-cell clusters in suspension, including the T cell-comprising cell-cell complexes, are obtained from the sample that was obtained from a subject. These cell-cell complexes are present in the sample material that was obtained from the subject. This is opposed to any method that (artificially) induces formation of cell-cell complexes/clusters in vitro (e.g. by in vitro cultivation or by purposely admixing different, for example in vitro prepared, cell types, in other words, opposed to any artificial formation of the complexes, outside the sample. In the invention, the T cell-comprising cell-cell complexes are cell-cell complexes that originate from the sample obtained from the subject.

Therefore, in embodiments of the invention, there is provided isolating a cell-cell complex comprising a T cell, and/or a T cell comprised in such cell-cell complex, from a sample obtained from a subject. The skilled person will understand that any embodiment pertaining to any method, use or composition disclosed herein likewise is applied to such embodiment of the invention.

In certain embodiments, two or three of a tumor tissue sample, a blood sample and/or a lymph node sample are obtained from the subject and used in the method according to the invention.

In a next step of the method of the invention, the dissociated cells obtained from the tumor tissue sample and/or the treated blood sample and/or the lymph node sample are used to identify therein a cell-cell complex comprising a T cell, and/or a T cell comprised in the cell-cell complex. Within the context of the current invention, such T cell comprising cell-cell complexes are understood to refer to a complex or cluster of at least two cells (including heterotypic clusters), and wherein at least one cell is a T cell, and preferably, wherein the T cell in the cell-cell complex is conjugated, preferably via TCR-antigen binding, to at least one other cell, preferably expressing the antigen for the cognate TCR present in the T cell. Examples include the cell:cell doublets are referred to herein elsewhere. Within the context of the current invention, such T cell-comprising cell-cell complexes may, for example, be identified by being double positive for a T cell-specific marker and for a marker that is specific for the at least one other cell in the cell-cell complex, for example for an APC-specific marker, e.g. DC-specific marker in case of an APC (e.g., DC), or, for example for a tumor-specific marker in case of a tumor cell that is present in the cell-cell complex, for example as judged by flow cytometry.

The skilled person is well-aware of methods to identify and/or isolate the T cell comprising cell-cell complex, for example using methods as described herein.

Suitable method for identifying and/or isolating the T cell comprising cell-cell complex, and, for example separating these from any single cells present in the sample or from any aggregation of cells, or any other cluster of cells but not comprising a T cell include, but are not limited to, methods based on physical, electrical, chemical or biological characteristics (or combinations) of the T cell comprising cell-cell complexes, methods using antibodies, including, for example a (bispecific) anti (nano) body, that recognizes MHC and TCR on two different cells that are interacting, or using different markers wherein at least one marker is specific for T cells, and, preferably, wherein at least one marker is specific for a non-T cell that is comprised in the cell-cell complex, or any combination of such methods.

As will be understood by the skilled person, once a T cell-comprising complex is identified and/or isolated, subsequently the T cell comprised in the T cell comprising complex may be identified and/or isolated as well, again using methods well-known to the skilled person.

Likewise, the skilled person understands that any other cell that is, next to the T cell, comprised in the T cell-comprising cell-cell complex, may after identifying and/or isolating the T cell-comprising cell-cell complex, be identified and/or isolated, using methods well-known to the skilled person.

In some embodiments of the method of the invention, the cell-cell complex is a T cell-tumor cell complex and/or wherein the cell-cell complex is a T cell-Antigen-presenting cell (APC) complex, preferably wherein the APC is a dendritic cell (DC), a B-cell and/or a macrophage. Preferably, the cell-cell complex is a T cell-tumor cell complex, and wherein the T cell is conjugated to the tumor cell via TCR-antigen binding.

In some embodiments the cell-cell complex is a T cell-tumor cell complex and wherein the tumor cell is no longer intact, for example wherein the tumor cell has at least partially been degraded, for example as the consequence of the cytotoxic activity of the T cell conjugated thereto. The skilled person will understand that such degraded tumor cell may be identified using, for example, an antibody or the like directed to a cognate binding partner in the tumor cell and, for example, wherein the cognate binding partner is only accessible to the antibody or the like when the tumor cell is at least partially degraded.

As will be clear to the skilled person, the method of the invention thus not only allows for the identification and/or isolating of a T cell that is comprised or expressed in the T cell-comprising cell-cell complex, but independently or simultaneously allows for the identification of a tumor cell and/or an APC that is comprised in (the same) T cell-comprising cell-cell complex.

Therefore, also provided is for the method of the invention further comprising identifying and/or isolating a tumor cell and/or an APC comprised in the cell-cell complex.

In some embodiments, the cell-cell complex comprises more than one T cell and/or more than one tumor cell and/or more than one APC. For example, in some embodiment the cell-cell complex comprises one T cell and one tumor cell, or one T cell and two tumor cells, or one tumor cell and two T cells, or one or more T cells, one or more tumor cells, and one or more APCs, for example one or more DCs. The skilled person thus understands that the cell-cell complex, including wherein the cell-cell complex is a T cell-tumor cell complex or a T cell-APC complex, may comprise additional cells, for example one or more APCs may be present in a T cell-tumor cell complex. In other words, where herein reference is made to a T cell-tumor cell complex, such complex may also comprise an APC. In other words, where herein reference is made to a T cell-APC complex, such cell-cell complex may also comprise a tumor cell. In those embodiments wherein the cell-cell complex comprises more than one T cell, the more than one T cell may be the same or different T cells.

In some embodiments the ratio in the cell-cell complex of T cell:non-T cell is between 1:10 and 10:1, preferably between 1:5 and 5:1, even more preferably between 1:2 and 2:1, most preferably 1:1. Herein the ratio refers to the ratio in the number of cells. In some embodiments the cell-cell complex comprises 50 cells or less, 40 cells or less, 30 cells or less, 20 cells or less, or 10 cells or less. In a preferred embodiment the cell-cell complex comprises between 2 and 20 cells, or between 2 and 10 cells.

As discussed herein elsewhere, it was found that with the method of the invention T cells may be identified and isolated that are tumor-reactive cytotoxic T cells. Therefore, in some embodiments there is provided that the T cells are tumor-reactive cytotoxic T cells.

In a preferred embodiment of the invention, the method comprises freezing the sample after step (ii), followed by thawing of the sample before step (iii). Although the method of the invention may be performed using a fresh sample that has been obtained from the subject, it was surprisingly found that the method of the invention may also be performed using a sample that has been frozen after step (ii) and subsequently be thawed before step (iii).

The skilled person is well aware of methods for freezing the sample obtained from the subject. As will be understood by the skilled person, such method may include the use of freezing media that allow the cells to remain alive when frozen and subsequently thawed. Typically, such freezing media comprises polyethylene glycol, DMSO and/or other comparable anti-ice crystal forming compounds.

In another preferred embodiment of the invention, the method comprises contacting or treating the sample with a cell death inhibitor, preferably an apoptosis inhibitor, preferably wherein the apoptosis inhibitor is a caspase inhibitor, preferably selected from ZVAD (zVAD-fmk), VAD, YVAD, ZYVAD-fmk, XIAP, BIR, clAP-1, clAP-2, Quinoline-Val-Asp-Ch2-O-Ph (QVD), or BAF, preferably wherein the contacting is at least in step (i) and/or (ii). Although not necessary for the invention, the use of such cell death inhibitors may further improve the method of the invention. These and other cell death inhibitors and/or apoptosis inhibitors are well-known to the skilled person and the use thereof has been described in various scientific and patent documents.

In some embodiments, the method comprises performing a disaggregation step on the sample before, during or after step (ii). The skilled person understands how to perform such disaggregation step.

In some embodiments of the invention, dissociating the tumor tissue sample and/or the a lymph node sample in step (ii) comprises enzymatic treatment of the tumor tissue sample and/or a lymph node sample to dissociate the cells in the tissue, preferably wherein at least a DNAase and/or a collagenase is used, and/or wherein a cell death inhibitor, for example an apoptosis inhibitor, is used, and, preferably, wherein the treatment is for a period of between 5-60 minutes, for example 10-40 minutes or 25-35 minutes, at a temperature between 30-40 degrees Celsius, for example 36-38 degrees Celsius. These and other methods of enzymatic dissociation of the tumor tissue sample and/or a lymph node sample are well-known to the skilled person.

In some embodiments of the invention, the lysing or removal of erythrocytes from the blood sample in step (ii) comprises treatment of the blood sample with a Red Blood Cell Lysing Buffer, preferably for a period of 5-25 minutes, for example 10-15 minutes, at a temperature of between 15-25 degrees Celsius. These and other methods of the lysing or removing erythrocytes from the blood sample are well-known to the skilled person.

In some embodiments of the invention, in step (iii) the cell-cell complex comprising a T cell is identified and/or isolated by using a T cell specific marker. By using T cell-specific markers, T cells that are comprised in the cell-cell complexes according to the invention may be identified and distinguished from T cells that are not present in the cell-cell complexes according to the invention, but, for example, are present as single (non-conjugated) cells. Within the context of the current invention a T cell-specific marker is a marker that is specific for a T cell relative to another cell that is comprised in the cell-cell complex, for example relative to a tumor cell or APC. In other words, a T cell-specific marker allows to identify a T cell. In a comparable manner, within the context of the invention should a tumor specific marker and/or an APC-specific marker be understood as being able to identify a tumor cell and an APC, respectively.

Therefore, in some other embodiments of the method of the invention, in step (iii) the cell-cell complex comprising a T cell is identified and/or isolated by using a T cell-specific marker and a tumor cell-specific marker, and/or by using a T cell-specific marker and an APC-specific marker, and/or by using a T cell-specific marker, a tumor-specific marker and an APC-specific marker, and preferably wherein the cell-cell complex comprising a T cell is further identified and/or isolated by determining T cell activation of the T cell comprised in the cell-cell complex, preferably using a T cell activation-specific maker, preferably wherein the T cell activation specific maker is CD69, or any other marker, or genetic signature, associated with T cell activity (including, for example a marker in the tumor cell indicative of T cell induced apoptosis and/or cell death of the tumor cell).

Also provided is for the method of the invention wherein the method further comprises determining T cell activation of the T cell comprised in the cell-cell complex, preferably wherein T cell activation is determined using CD69 as a marker.

In some embodiments, the T cell is a CD8+ T cell and/or a CD4+ T cell, preferably the T cell is a CD8+ T cell. In some embodiments, therefor, T cells may be identified and/or isolated by using a CD8+ T cell specific marker, a CD4+ T cell specific marker, or a CD8+ T cell specific marker and a CD4+ T cell specific marker. For examples in some embodiments cell-cell complexes are identified that only comprise, as T cell, a CD8+ T cell. For examples in some embodiments cell-cell complexes are identified that only comprise, as T cell, a CD4+ T cell. For examples in some embodiments cell-cell complexes are identified that comprise, as T cell, both a CD4+ T cell and a CD8+ T cell. The presence of CD4 T cells may provides important support for the killing activity of CD8 T cells, making such CD8+ and CD4+ comprising cell-cell complexes of interest in the context of the current invention.

In some embodiments, the cell-cell complexes comprising a T cell are identified and/or isolated using cell sorting, flow cytometry, and/or imaging flow cytometry. The skilled person is well-aware how to perform such methods within the context of the current invention, for example, as described herein.

In some embodiments of the method of the invention, the method further comprises identifying the T cell receptor that is comprised or expressed in the isolated T cell. Again, the skilled person is well-aware of methods for the identification of the T cell receptor that is expressed in the T cell that was comprised in the T cell comprising cell-cell complex that was identified and/or isolated with the method of the invention. The skilled person also understands that the TCR thus obtained or identified may be engineered and introduced in other T cells to provide for engineered T cells that are useful in the treatment of cancer, for example, and preferably in the subject from which the sample was (initially) obtained. Treatment of a subject or patient with the thus obtained engineered T cell is also contemplated by the current invention.

According to other embodiments of the invention, also provided is for the in vitro expansion of the isolated T cells, APC and/or tumor cells. The skilled person is well-aware of suitable methods for the further expansion of the isolated T cell, APC and/or tumor cell. For example, T cells may be expanded using so called REP methods (rapid expansion protocols), well known to the skilled person. As will be understood by the skilled person, such expanded T cells may subsequently be used in the treatment of cancer, for example, and preferably, of the cancer in the subject from which the sample was (initially) obtained, for example in adoptive T cell therapy (ACT), for example but not limited to TIL therapy. The same may apply to the expanded APCs. At the same time, the isolated T cell, tumor cell and/or APC may be used to prepare, respectively, a T cell line, tumor cell line and/or APC cell line from the subject.

According to another aspect of the invention there is provided for a method of identifying a TCR useful in the treatment of a subject, a T cell useful in the treatment of a subject and/or a APC useful in the treatment of a subject and/or a tumor cell, the method comprising performing the method according to the invention to identify in step (iii) from the sample obtained after step (ii) the cell-cell complexes comprising a T cell, and wherein the method further comprises identifying/and or isolating the T cell and/or the APC and/or the tumor cell from the cell-cell complex, and/or identifying the TCR expressed in the isolated T cell.

As will be understood by the skilled person, within the context of the current invention the one or more TCR may be identified during each step of the method of the invention. For example, the TCR may be identified immediately after isolation of the clinical sample, or after one or more steps in the method of the invention. The TCR may also be identified after expansion of the T cells as described herein (e.g. using a REP (Rapid expansion) protocol. The TCR may also be identified after co-culturing with tumor cells or after T cell activity measurement, or after any step that further enriches for T-cells/TCRS have (higher) anti=tumor activity.

Also provided is for the T cell, APC cell, tumor cell or TCR obtained with the method according to the invention.

With respect to the method of the invention, preferably the method does not include before step (iii) a step of in vitro culturing of the cells in the sample obtained from the subject.

Also provided is for a T cell, APC or TCR for use in the treatment of cancer in a subject, wherein the treatment comprises identifying and/or isolation of the T cell, APC or TCR with the method according to the invention, and
(i) expanding the T cell and/or APC and administering the expanded T cell and/or APC to the subject, preferably wherein the sample was obtained from the subject; and/or
(ii) identifying the TCR comprised or expressed in the T cell, preparing an engineered T cell using the identified TCR, expanding the engineered T cell, and administering the expanded engineered T cell to the subject, preferably wherein the sample was obtained from the subject.

Finally, also provided is for a method of identifying cell useful in the treatment of a subject, preferably a method of identifying a TCR useful in the treatment of a subject, a T cell useful in the treatment of a subject, and/or an APC useful in the treatment of a subject, the method comprising performing the method according to any of the previous aspect and embodiments to identify and/or isolate in step (iii) from the sample obtained after step (ii) the cell-cell complexes comprising a T cell, wherein the method further comprises:
identifying and/or isolating a cell from the cell-cell complex, preferably identifying/and or isolating the T cell and/or the APC from the cell-cell complex;
performing one or more of a gene expression analysis, and an omics analysis, preferably proteomic analysis, of the identified and/or isolated cell from the cell-cell complex, preferably of a T cell and/or of a APC identified/and or isolated from the cell-cell complex;
comparing the results of the one or more of a gene expression analysis, and omics analysis, with the results of the gene expression analysis and/or omics analysis performed on cell, preferably on a T cell and/or on an APC, of the isolated sample obtained after step (ii), wherein said cell, preferably said T cell and/or APC, is not comprised in a cell-cell complex,
wherein one or more of a differentially expressed gene or differential omics is used as marker to identify a cell, preferably a T cell and/or an APC, to be expanded and/or a cell, preferably a T cell useful in the treatment of a subject and/or preferably an APC useful in the treatment of a subject, and/or TCR useful in the treatment of a subject.

Although preferably T cells and/or APC identified and/or isolated from the cell-cell complex are preferred to be expanded and/or for use in the treatment of the subject, other cells, such as other immune cell types or tumor cells can be used.

Herewith provided is furthermore a method for identifying markers for the selection of a tumor reactive cell, preferably a tumor reactive T cell and/or APC, in an isolated sample of a subject, wherein the subject is diagnosed with cancer and/or is suspected of having cancer, the method comprising performing the method according to any one of the previous aspect and embodiments to identify and/or isolate in step (iii) from the sample obtained after step (ii) the cell-cell complexes comprising a T cell, wherein the method further comprises:
identifying/and or isolating the cell, preferably the T cell and/or the APC, from the cell-cell complex;
performing one or more of a gene expression analysis and an omics analysis, preferably proteomic analysis of the cell, preferably of the T cell and/or of the APC from the cell-cell complex;
comparing the results of the one or more of a gene expression analysis, and omics analysis, with the results of the gene expression analysis and/or omics analysis performed on a cell, preferably on a T cell and/or on an APC, of the isolated sample obtained after step (ii), wherein said cell, preferably said T cell and/or APC, is not comprised in a cell-cell complex
wherein one or more of a differentially expressed gene or differential omics is used as marker for the selection of a tumor reactive cell, preferably a tumor reactive T cell and/or APC in an isolated sample of a subject.

The gene expression analysis encompasses determining by conventional techniques which genes are expressed in a particular cell or cells and at a particular development and/or specialization cell state. Conventional techniques include, without limitation, the detection of messenger RNA and/or the detection of proteins in a cell. For the analysis of differentially expressed genes between two cell types, in the invention between T cell and/or APC comprised in a cell-cell complex and T cell and/or APC not comprised in a cell-cell complex, also conventional techniques apply, including for example informatic tools for the analysis of high amounts of data.

Omics analysis encompasses genomics, proteomics, metabolomics, metagenomics, phenomics and transcriptomics. Omics aims at the collective characterization and quantification of pools of biological molecules that translate into the structure, function, and dynamics of an organism or organisms. In embodiments of the invention, the omics is genomics. In embodiments of the invention, the omics is proteomics. In embodiments of the invention, the omics is metabolomics. In embodiments of the invention, the omics is metagenomics. In embodiments of the invention, the omics is phenomics. In embodiments of the invention, the omics is transcriptomics.

The foregoing description of the specific embodiments will so fully reveal the general nature of the invention that others can, by applying knowledge within the skill of the art (including the contents of the references cited herein), readily modify and/or adapt for various applications specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It will be understood that all details, embodiments, and preferences discussed with respect to one aspect of embodiment of the invention is likewise applicable to any other aspect or embodiment of the invention and that there is therefore no need to detail all such details, embodiments, and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

### Example 1

### Introduction, Results, and Conclusions

The current invention is based on studies on physical and functional interactions between human tumor cells, APC and T cells in vitro and in vivo. The inventors realized that functional T cell:tumor cell and T cell:APC interactions are key in patients for an appropriate immune (therapeutic) response, and hypothesized that stable physical interactions between T cells (through their specific TCRs) and either tumor cells or APC (through the (neo) antigens they present) may serve as a critical functional feature that can be used to specifically identify (and/or enrich) and isolate cancer-killing T cells from patients.

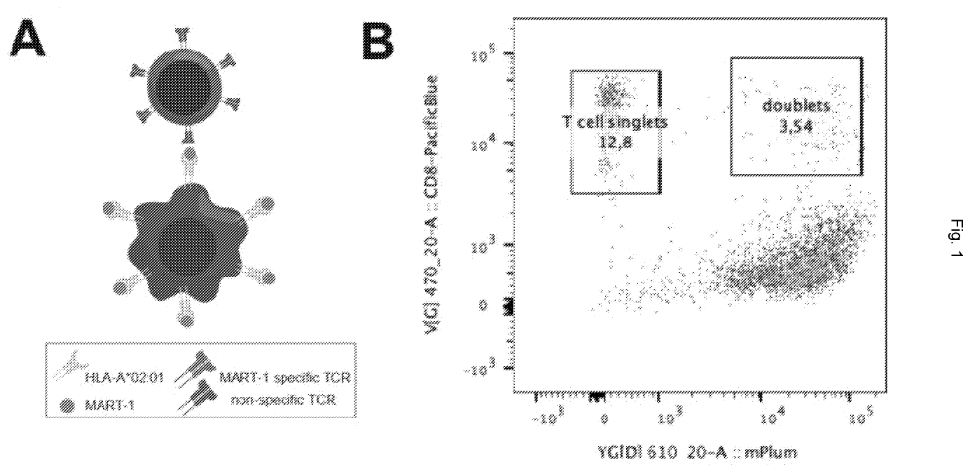

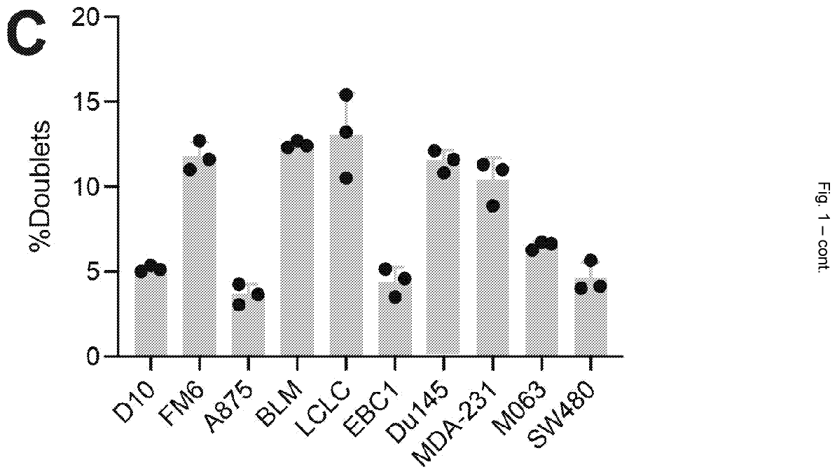

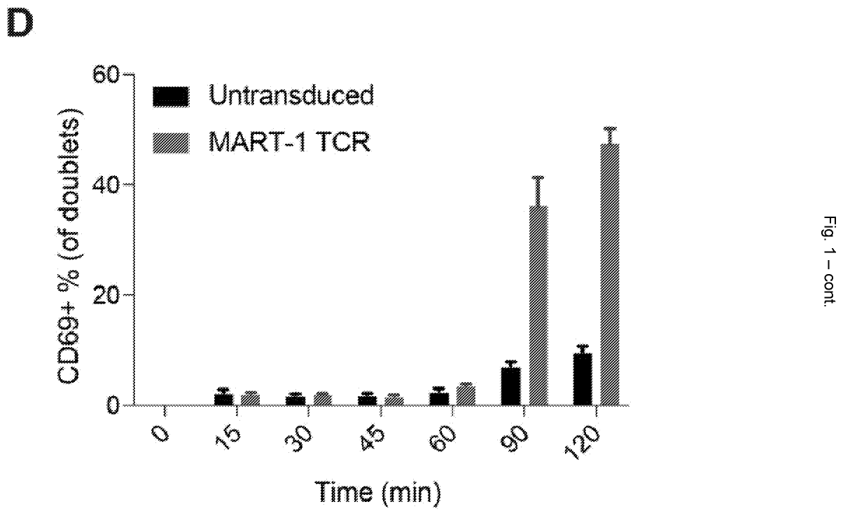

To study physically interacting T cells and tumor cells, initially use was made of an in vitro T cell:tumor co-culture model: the tumor cells were genetically engineered to co-express HLA-A*02:01 and a MART-1 antigen. On the other hand, T cells were transduced with a MART-1-specific TCR. When tumor and T cells are co-cultured, T cells are able to recognize tumor cells and trigger tumor cell apoptosis (FIG. 1A). When co-culturing the two pre-labelled cell types, stable cell:cell doublets (herein also referred to as cell-cell complexes, or T cell-comprising cell-cell complex or clusters, i.e., a complex of at least two cells, and wherein at least one cell is a T cell, and preferably wherein the T cell is conjugated (via TCR-antigen binding) to at least one other cell (expressing the antigen, for example a tumor cell and/or a APC) can be identified, as judged by a double-positive subpopulation by flow cytometry (FIG. 1B). Doublet formation was evaluated in a panel of cancer cell lines. All cells were able to form doublets with T cells to different extents (FIG. 1C).

To determine any functionality of these doublets, we evaluated T cell activation within the doublets in a time course. We observed that the CD69 activation marker was upregulated after 90 min of the interaction in matched T cells (FIG. 1D). Importantly, this was almost absent in unmatched T cells, demonstrating specific activation.

Next, we assessed whether these doublets formed with equal stoichiometry (1:1 T cell:tumor cell), or instead represented either multicell clusters or contained single cell types (either tumor or T cell). Therefore, we used Imagestream®, a flow cytometer equipped with a microscope. We observed that at least 80% of doublets corresponded to 1:1 T cell:tumor cell doublets (80% for D10 and 82% for A875 melanoma cell lines; FIG. 1E).

In the above in vitro model, the TCR used was a high affinity MART-1 TCR. However, in tumors in patients there are TCRs with different affinities. To assess how affinity influences the doublet formation and activation, we also made use of a low affinity MART-1 TCR (DMF4). We observed that the use of a low affinity TCR decreased the number of doublets formed, as well as the degree of activation of the T cells within those doublets compared with high affinity TCR (1D3) (FIG. 1F). However, also low affinity TCR (and/or low activation of the T cells within the T cell comprising cell-cell complexes), can still be identified.

In addition, in in vivo patient tumor settings there are heterogeneous T cell populations with different TCRs, including many that do not recognize TAAs. To investigate whether this affects cluster formation (i.e., the formation of the T cell-comprising cell-cell complexes), we designed an experiment in which we mixed MART-1-specific and unspecific T cells to determine which ones will preferentially form doublets with MART-1-expressing T cells. We commonly observed an enrichment of MART-1-specific T cells, compared to untransduced T cells, in doublets (FIG. 1G). These data indicate that in a competitive context, such as exists in patient tumors in vivo, the tumor: T cell doublets may be enriched for T cells with specific TAA reactivity.

Together, these data suggest that, using in vitro cultivated cells, a TAA-TCR match establishes a relatively stable interaction, which allows not only for the formation of tumor: T cell doublets, but also leads to more robust T cell activation.

However, as will be understood by the skilled person, in a tumor in a patient, there are many additional factors, such as the presence of various types of cells, the presence of heterogenous populations of T cells, with varying strength of interaction with the tumor cells, the presence of heterogenous population of tumor cells, with different tumor cells expressing different (combinations of) antigens, the presence of additional immune cells, including, for example, dendritic cells, and the, in general, hostile, tumor microenvironments, that make it unpredictable, or even unlikely that in tumor or blood material obtained from such patient (or subject) T cells comprising cell-cell complexes can be identified and/or isolated (and/or enriched) in a useful manner, and wherein the T cells comprised in such T cell-comprising cell-cell complex may be a tumor-reactive T cell that is useful in the treatment of the cancer of the subject.

We therefore set out to establish whether also in cancer patients, stable complexes between tumor cells and T cells specifically recognizing those tumor cells may exist, and, more importantly, may be identified and/or isolated from a tumor and/or blood sample and/or a lymph node sample obtained from the cancer patient, and subsequently allow for specific identification, isolation and/or enrichment of TAA-specific killer T cells. We tried to identify this in clinical samples.

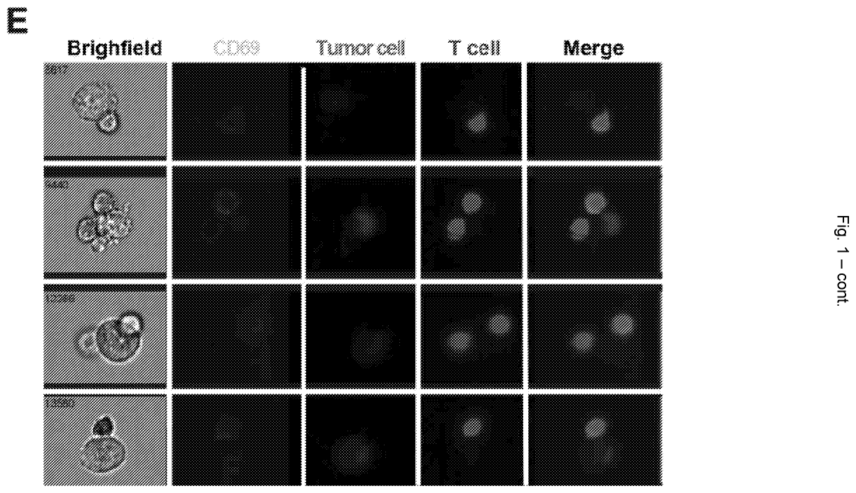

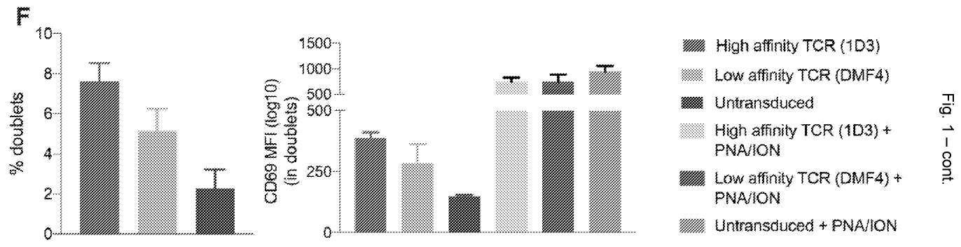

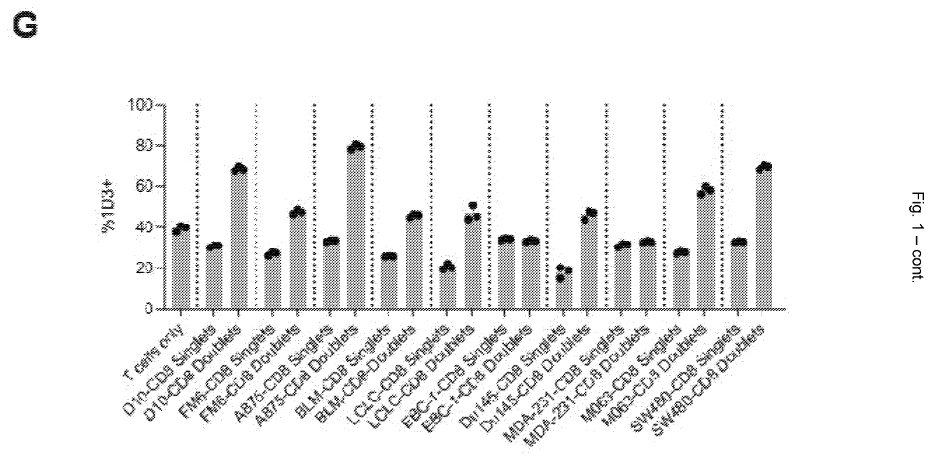

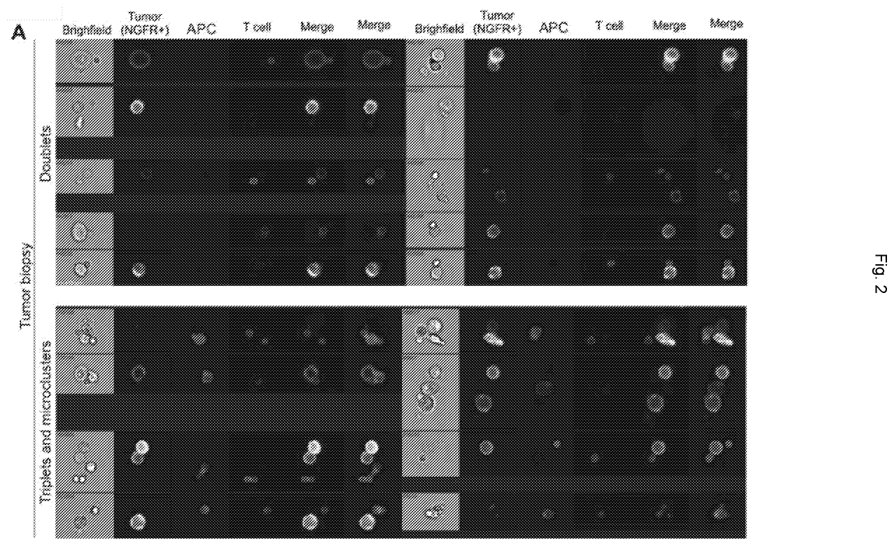

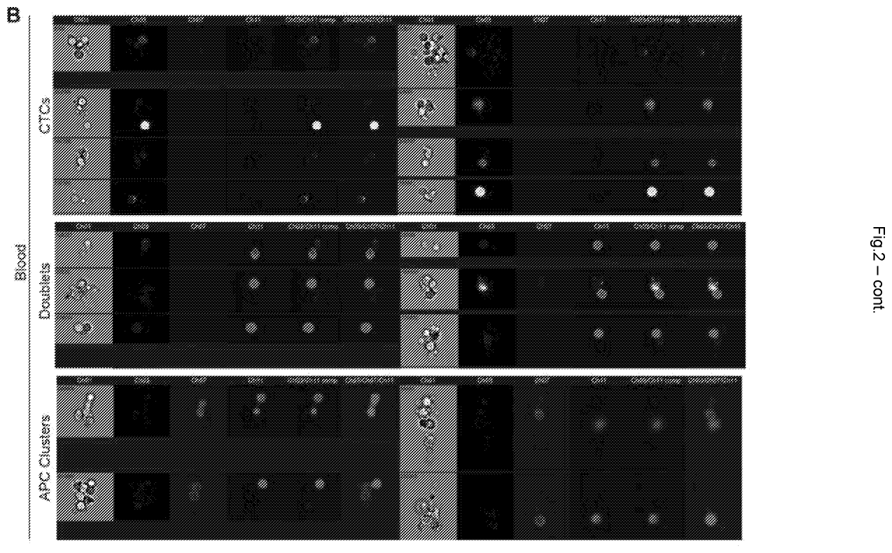

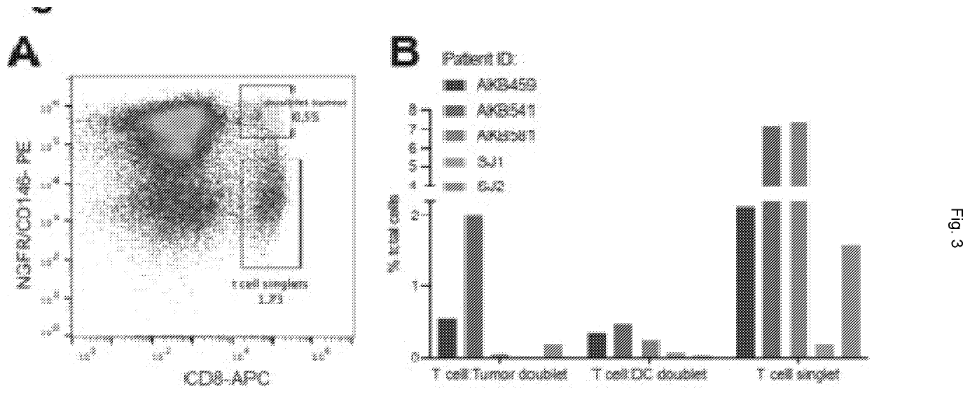

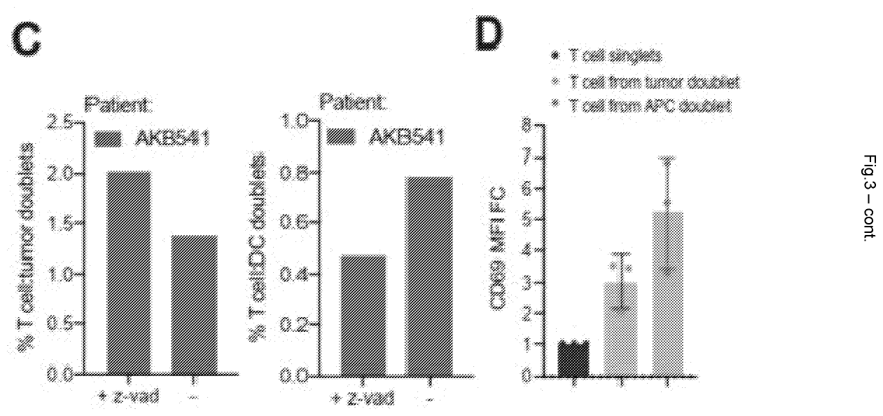

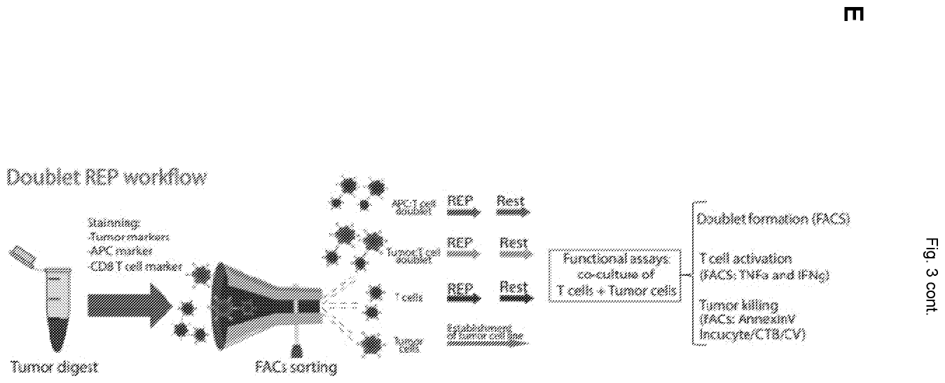

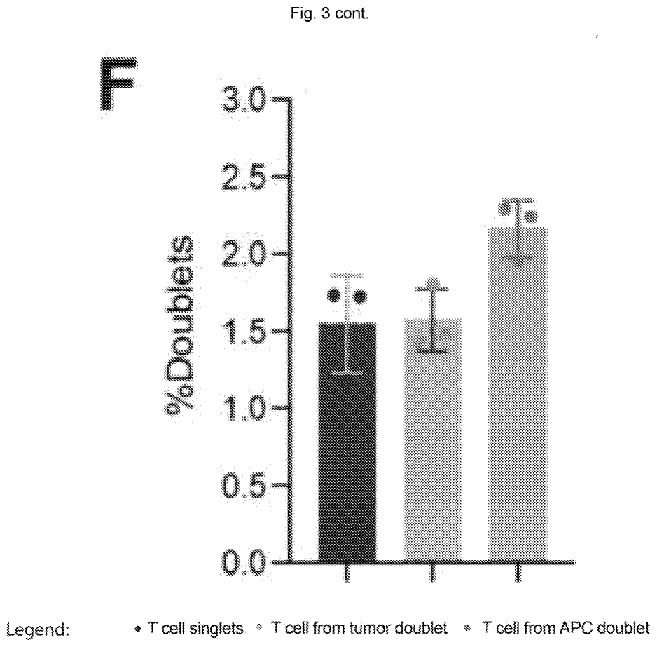

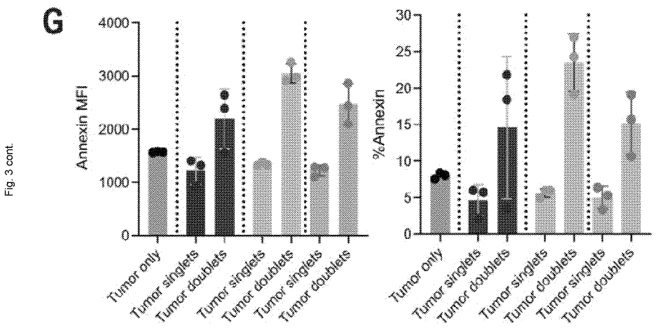

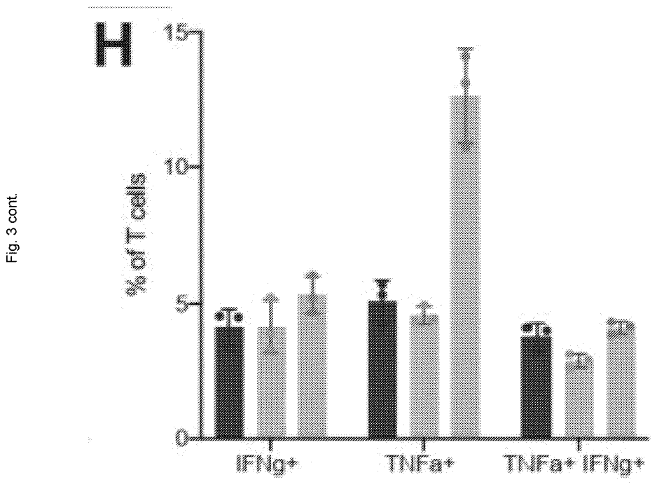

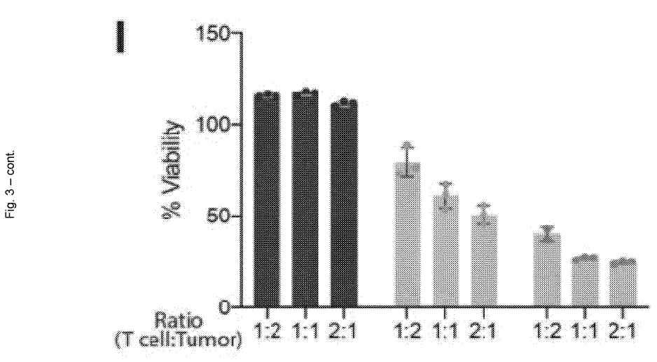

Patient material was obtained as tumor digest or PDTFs (patient-derived tumor fragment (PDTF) platform, in collaboration with Daniela Thommen) (Voabil et al., 2021). In both cases, a short digestion protocol was performed of the sample prior to freezing it down. Importantly, we included a cell death inhibitor/apoptosis inhibitor, either ZVAD or QVD, to freeze or prevent the apoptosis process, in order to maintain (partly) intact tumor cells in association with specific killer T cells. After thawing, a staining protocol was conducted, and samples were analyzed by both flow cytometry and Imagestream®. In addition to the T cell:tumor interaction, we included in these set of experiments stainings for T cell:APC interactions. A final staining protocol may, for example, include tumor cell markers (for example, but not limited to, NGFR, CD146), T cell markers (for example, but not limited to, CD8, CD3, CD69) and APC markers (for example, but not limited to, CD11c). An alternative panel of markers may be one that also assess for synapse formation, T cell activation and antigen presentation (for example, but not limited to, TCR, CD103, CD86, HLADR, AnnexinV, etc.). We made the following observations: with Imagestream® analysis, we observed 1:1 T cell:tumor doublets as well as T cell:APC doublets (FIG. 2A), and commonly even triplets or larger T cell clusters. Strikingly, we observed commonly T cells in complex with disintegrated tumor cells, which were clearly no longer intact and likely no longer viable, but apparently maintaining sufficient integrity for recognition by the tumor cell marker-staining antibody, and capable of maintaining a physical interaction with the T cell that was sufficiently strong to survive, for example, a freeze-thaw step and experimental stress. All this was observed in clinical samples that had undergone a freeze-thaw cycle. We identified with Imagestream® analysis circulating tumor cells (CTCs) in the blood of patients and were also able to identify several APC:T cells and even some rare CTC:T cell clusters (FIG. 2B). We identified (n=3 different patient samples) around 0.5% of T cells in complex with either a tumor cell, an APC or both (FIGS. 3A and 3B). We observed that the inclusion of cell death inhibitor, for example an apoptosis inhibitor, such as ZVAD, resulted in appr. 30% increase in the number of tumor: T cell complexes. This suggests that at least a proportion of the cellular aggregates involve killer T cells with actual and specific antitumor activity) (FIG. 3C). We observed that T cells in doublets with either tumor cells or APCs were more activated, as measured by CD69 expression, than T cell singlets (FIG. 3D). To show the functional activity of T cells in such cell-cell complexes, we sorted patient-derived CD8 T cells that were in tumor: T cell or APC:T cell clusters, as well as single T cells. We expanded those using a REP-like protocol and performed functional experiments using those REP-ed cells. We also sorted tumor cells of the same patient and established a patient-derived tumor cell line (FIG. 3E). By co-culturing the cell line and the expanded T cells we were able to show that T cells derived from APC doublets form more secondary doublets and produced more TNFa when in contact with tumor cells compared to T cells derived from singlets. In addition, T cells derived from APC and tumor cell-cell clusters are slightly better at inducing tumor cell Annexin V in short-time co-cultures (FIG. 3F-H). In long-term co-cultures, we observed a marked increase of tumor killing in the cultures with T cells derived from APC or tumor doublets compared to T cells derived from singlets (FIG. 3I, J). This supports our idea that T cells clustered with tumor cells and/or APC in vivo are enriched for T cells with specific anti-tumor activity, as judged by the several secondary functional assays.

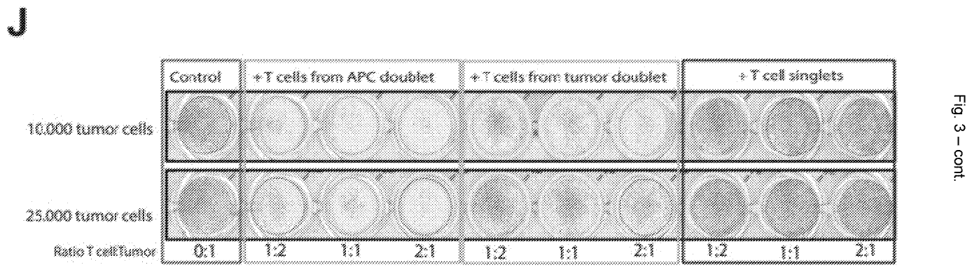

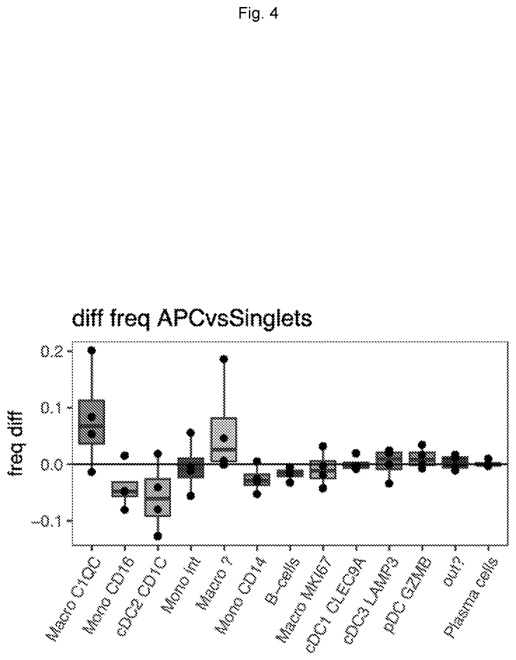

With the aim of also further characterizing the isolated from tumor cell:cell complexes, APC cells were also analyzed to determine differing phenotypes within the APC in clusters (APC:T cell clusters) versus singlets APCs. Data are illustrated in FIG. 4, wherein higher bars indicate a higher frequency of that phenotype in APCs from clusters. Each dot represents data from an individual patient.

Results herewith obtained with melanoma tumors were also observed in samples isolated from other type of tumors, including a non-small cell lung (NSCLC) tumor digest and a penile tumor digest.

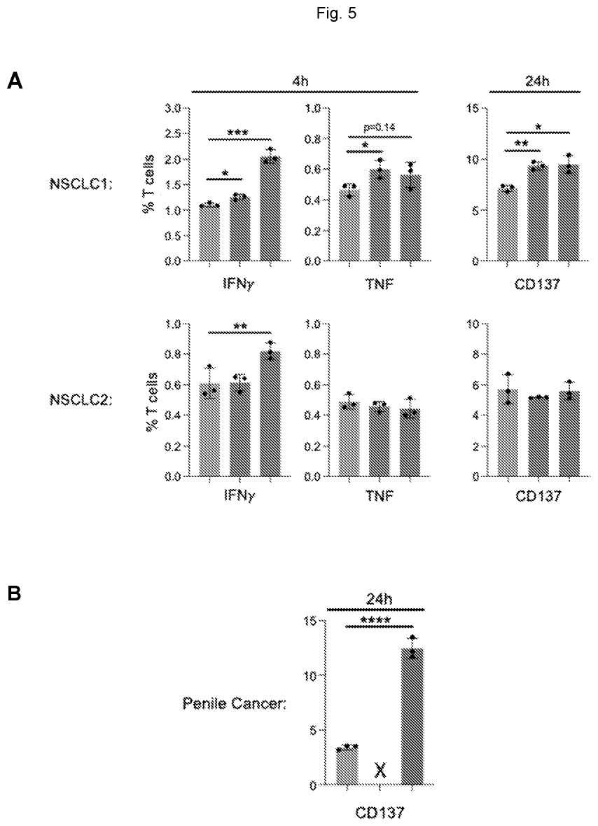

Expanded (as previously disclosed) T cells derived from T cell:tumor cell and T cell:APC (i.e., heterotypic clusters) in non-small cell lung cancer and penile cancer patients, showed higher TNF, IFNy production and CD137 upregulation in T cells from complexes (clusters) than in T cells from singlets (FIG. 5 (A) and (B)).

In conclusion, our results demonstrate the common existence in cancer, of smaller (doublets) or larger T cell clusters (3 or more cells) that contain tumor cells and/or APCs, which have sufficient stability and/or avidity to resist a freeze-thaw step and experimental handling including flow cytometry. T cells were found in complex with either intact or disintegrated tumor cells. Based on the ZVAD treatment and other results described above, our data suggest that all these T cells are enriched for T cells equipped with TAA-specific TCRs. We thus propose that these T cell clusters are formed based on functional interactions that may be used to identify killer T cells with specific TAA antitumor activity.

Furthermore, we propose that not only intact clusters of T cells, tumor cells and APC serve as a highly valuable source to develop immunotherapy, but also (or particularly) clusters involving T cells and disintegrated tumor cells. Specifically, the latter may be used as a powerful phenomenon and/or biomarker to identify killer T cells and their corresponding TCRs. In this respect, one may use an intracellular tumor marker (that is not accessible for antibody staining for intact tumor cells), such as S100 for melanoma cells or SOX2 for lung cancer cells for the specific identification of these conjugated active killer T cells. The thus obtained TAA-specific TCRs may be used for engineered TCR T cell therapies.

In addition to the use of TAA-specific TCRs, we believe that the current invention offers significant advantages to adoptive T cell therapy (ACT), for example but not limited to TIL therapies and may be used in such therapies (for example, after expansion of the T cells). As few as hundreds of specific and active T cells have been demonstrated to elicit robust antitumor responses in mice (Purcarea et al., 2022). The number of T cells necessary for this effect is highly dependent on the avidity of the TCR for the tumor antigen. Interestingly, not always the most abundant TCR has the highest avidity and those high avidity T cells are usually present at low frequencies in adoptive T cell therapy (ACT), for example but not limited to TIL therapies. Using our protocols, we could specifically expand this population and reinfuse into the patients. This invention is thus believed to have at least the following advantages over state-of-the-art protocols: Because the invention is based on functional parameters, notably cell:cell interactions and/or T cell antitumor activity (as witnessed by dying tumor cells attached to T cells), it is believed that the invention leads to a highly significant enrichment of specific antitumor T cells. Furthermore, we believe that this invention will have a beneficial impact on duration of the expansion protocol (e.g., fewer cells necessary for re-infusion, shorter turnaround time), toxicity and clinical outcome.

Another direct application of the herewith illustrated results is the use of T cells or APC from the T cell comprising complexes to determine the differential gene expression in relation to the T cells or APC as singlets in the isolated patient sample. The differentially expressed genes are then markers that help to identify the cells to be enriched (i.e., expanded) for use in therapy.

We demonstrate that T cells derived from these T cell comprising cell-cell complexes are functionally distinct in that they markedly outperform single T cells in exerting antitumor activity. T cells derived from these T cell comprising complexes reside also in different cell states. These profiles are typically seen for tumor-reactive T cells associated with exhausted and proliferative states. Most of the studies focused on the identification of tumor-reactive T cells proceed by using cell surface markers, including CD39 and PD-1. The procedure proposed according to the invention is different, in that independent of these markers, we identify and isolate a subpopulation of T cells (preferably CD8+ T cells and/or CD4+ T cells) displaying high tumor reactivity. More importantly, this represents a group of highly valuable cells that is excluded from common cell sorting procedures applied in clinics and in the prior art.

### Material and Methods of Example 1

### In Vitro PoC

### Isolation and Generation of MART-1 High and Low Affinity TCR CD8 T Cells

MART-1 TCR retrovirus (1D3) was produced in a packaging cell line as described previously (Gomez-Eerland et al., 2014). Alternatively, to compare MART-1 high and low affinity TCRs (1D3 and DMF4 respectively), retrovirus was produced by transfecting FLY-RD18 cells with 2.5 μg TCR-DNA plasmid using Xtremegene 9 transfection reagent. After 48 h, virus-containing supernatant was collected and centrifuged to remove cells. PBMCs were isolated from healthy donor buffycoats by density gradient centrifugation, after which CD8+ T cells were isolated using CD8 Dynabeads. CD8+ T cells were activated for 48 h in a 24-well plate that was pre-coated with aCD3 and aCD28 antibodies. After activation, CD8+ T cells were mixed 1:1 with MART-1 TCR retrovirus from the packaging cell line and spinfected on a retronectin coated non-tissue culture treated 24-well plate for 2 h at 2000 g. Alternatively, virus-containing supernatant from FLY-RD18 cells was transferred to retronectin-coated plates first, centrifuged for 90 minutes at 3500 g, 10 degrees Celsius, after which CD8 T cells were added. 24 h after transduction, T cells were harvested and cultured for 1 week before TCR expression was determined by flow cytometry using mouse TCR beta constant domain antibodies.

### Cell Lines and Co-Culture

All used tumor cell lines were transduced with lentiviral constructs encoding HLA-A*02:01, MART-1 and mPlum. Tumor cell lines were co-cultured with T cells transduced with MART-1-specific TCR in 96-well, non-tissue culture treated, V-bottom plates at the indicated ratios for 4 h. For the CD69 activation experiment, a time-course of 0-6 hours was performed. At end-point, plates were stained for flow cytometry or ImageStream analysis.

### Staining for Flow Cytometry and ImageStream

Co-cultures were washed with 0.1% BSA in PBS after which they were stained for 30 minutes on ice in the dark. After staining, cells were washed twice with 0.1% BSA in PBS before analysis. For flow cytometry, co-cultures were stained with CD8-Pacific Blue (1:100), mouse TCR beta-PE (1:100) and CD69-APC (1:100). For ImageStream analysis, CD8 T cells were stained with Cell Trace Violet (CTV) according to manufacturer's instructions before co-culture and CD69-PE (1:100) after co-culture. Doublets were identified being either mPlum-CD8 or mPlum-CTV double positive.

### Patient Samples PoC

### Tumor Digestion

To achieve single cell suspensions, tumors were digested in RPMI1640 medium. The tumor was cut in small pieces and pre-warmed enzymatic digestion medium (RPMI supplemented with pulmozyme (126 μg/mL), collagenase (1 mg/mL) and pan-caspase inhibitor (QVD 50 μM or Z-VAD 5 μg/mL)), was added and incubated at 37 degrees in a spinning rotor for a maximum of 30 minutes. The sample was passed through a 100 μm filter, washed and frozen in FBS+10% DMSO until further processing.

### Blood Processing

Full blood was taken in heparin tubes and 1 mL blood was mixed with 10 mL 1×red blood cell lysis buffer. This was incubated at room temperature for 15 minutes after which it was centrifuged for 5 min at 500 g. Supernatant was removed, and cells were immediately taken for subsequent staining for ImageStream or flow cytometry analysis.

### Staining for ImageStream, Flow Cytometry and Sort

Tumor digest was thawed and washed twice with RPMI, supplemented with 10% FBS and 1:1000 benzonase nuclease (purity>90%). Cells were washed an additional time with 0.1% BSA in PBS after which they were stained with an antibody mix (see Table 1) for 30 minutes on ice in the dark. After staining, cells were washed twice with 0.1% BSA in PBS before proceeding towards ImageStream, flow cytometry or sorting. Cells from blood were washed once with 0.1% BSA in PBS, followed by staining and washing as described for the tumor digest. For color compensation, beads stained with a single-color antibody were taken and processed at the same time.

TABLE 1
Antibody panels for sort and initial flow cytometry

| | |
|---|---|
| Panel 1 | Panel 2 |
| ImageStream | GEX and |
| REP sort | TCR-seq sort |
| Flow cytometry | Flow cytometry |

| | | | |
|---|---|---|---|
| NGFR-PE | 1:100 | NGFR-APC | 1:100 |
| CD146-PE | 1:100 | CD146-APC | 1:100 |
| CD8-APC | 1:100 | CD8-BUV805 | 1:50 |
| CD11c-BV421 | 1:40 | CD11c-BV421 | 1:40 |
| | | CD3-FITC | 1:50 |
| CD69-APC* | 1:100 | CD69-PE* | 1:100 |
| Near-IR | 1:1000 | Near-IR | 1:1000 |

*This antibody was added in flow cytometry experiments, but not used for sorting.

TABLE 2
Antibody panels for secondary co-culture experiments

| Panel 3a | Panel 3b | Panel 4a | Panel 4b |
|---|---|---|---|
| Sec. co- | Sec. co- | Sec. co- | Sec. co- |
| cultures | cultures | cultures | cultures |
| Cell | Intracellular | Cell | Intracellular |
| Surface | stain | Surface | stain |
| NGFR-PE | NGFR-PE | NGFR-APC | NGFR-APC |
| 1:100 | 1:100 | 1:100 | 1:100 |
| CD146-PE | CD146-PE | CD146-APC | CD146-APC |
| 1:100 | 1:100 | 1:100 | 1:100 |
| CD8-CTV | CD8-CTV | CD8-CTV | CD8-CTV |
| AnnexinV-PECy7 | 1:100 | AnnexinV-PECy7 | |
| 1:50 | | 1:50 | |
| CD69-APC | | CD69-PE | |
| 1:100 | | 1:100 | |
| PI | Near-IR | PI | Near-IR |
| 1:100 | 1:1000 | 1:100 | 1:1000 |
| | Intracellular: | | Intracellular: |
| | TNF-α-FITC | | TNF-α-PE |
| | 1:100 | | 1:100 |
| | IFN-γ-APC | | IFN-γ-FITC |
| | 1:100 | | 1:100 |

### Rapid Expansion Protocol

Tumor digest was thawed and stained with the antibodies in panel 1 of Table 1 as described above. Four populations were sorted using a BD Fusion flow cytometer: tumor singlets (NGFR+CD146+); tumor: CD8 T cell doublets (NGFR+CD146+CD8+); antigen presenting cell (APC): CD8 T cell doublet (NGFR-CD146-CD11c+CD8+) and CD8 T cell singlets (NGFR-CD146-CD11c-CD8+). Collected CD8 T cell populations were cultured in round-bottom 96-well plates which contained 45.000 35 Gray irradiated autologous feeder cells and 5.000 50 Gy irradiated EBV-JY cell line resuspended in RPMI medium supplemented with 10% human serum, 5% FBS, 300 IU/mL IL-2, 10 ng/ml IL-7, 10 ng/ml IL-15 and 0.8 μg/mL PHA. After 11 days, cells were collected and rested for at least 3 days in RPMI medium supplemented with 10% FBS and 100 IU/mL IL-2, before functional tests were performed. Collected tumor cells were cultured in tissue culture treated flat bottom plates in DMEM supplemented with 20% FBS and split when reaching confluency.

### Secondary Co-Cultures

### Doublet Formation and T Cell Activation

The rested CD8 T cell populations were labeled with cell trace violet (CTV) according to manufacturers instructions. Labeled CD8 T cells were co-cultured for 4 h with the established patient tumor cell line or tumor digest in a ratio of 1:4 (T cell:Tumor) in 96-well V-bottom plates. After 4 h, co-cultures were washed once with 0.1% BSA in PBS and stained with antibody mixes of panel 3 or panel 4 as described in Table 2. For intracellular staining, the FOXP3/Transcription Factor Staining Buffer Set was used according to manufacturer's instructions. Cells were analyzed for doublet formation (NGFR+CD146+CTV+), doublets containing dying cells (AnnexinV signal in NGFR+CD146+CTV+ cells) and T cell activation (TNF-α and IFN-γ in CTV+ cells) on a Fortessa flow cytometer.

### Tumor Cell Killing

10.000 or 25.000 tumor cell line and 20.000 or 50.000 tumor digest was seeded in 96-well flat bottom plates and allowed to adhere. CD8 T cells were added in three different ratios on day 0:1:2; 1:1; 2:1 (T cell:Tumor). On day 3, T cells were washed away, and tumor cells were re-challenged with 25.000, 50.000 and 100.000 T cells, respectively. On day 7, T cells were washed away, and tumor viability was assessed with Cell Titer Blue according to manufacturers instructions. Afterwards, plates were stained with a 0.1% crystal violet and 50% methanol solution for 1 h, air-dried and images were made of the cells remaining in the wells.

### Identification of APC Subsets by Single Cell RNA Sequencing

After integration of the scRNA-seq data sets, clusters identified as APC (based on expression of ITGAX, CD19, CD14) were subsetted in Seurat (version 4.3.0.1). Batch effect correction with Harmony (version 0.1.1), clustering and UMAP generation were performed as previously described on the subsetted object. Newly obtained clusters were excluded if they expressed high levels of tumor markers (MCAM, PMEL, MLANA) or if a large proportion of cells within the cluster contained TCR sequences, as an indication of doublets with tumor or T cells respectively. Remaining clusters were annotated based on marker genes determined by differentially expressed genes and on the average expression per cluster. APC specific markers were based on markers described in Cheng et al. 1 and Kapellos et al.2 Additionally, we used the classifier scGate (version 0.14) 3 with the TME HiRes model for identifying APC subsets.

### Example 2

Introduction, Results, and Conclusions. Heterotypic CD8 T Cell Clusters Isolated from Clinical Samples are Distinct and Enriched for Antitumor Activity

By means of this example it was further investigated whether tumor-specific CD8+ T cells could be isolated from clinical cancer specimens as heterotypic clusters. It was also investigated whether they exist in a different phenotypic state compared to T cell singlets, what their TCR clonality is, what their capacity is to produce cytokines and lastly, how active they are against autologous tumor cells.

Antigen-specific CD8+ T cells outcompete non-specific T cells in heterotypic tumor cell clusters

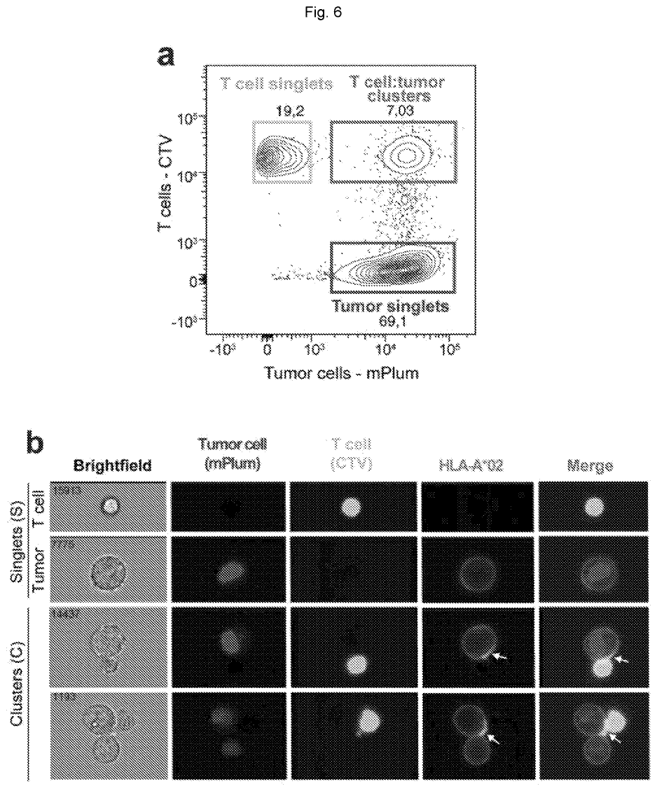

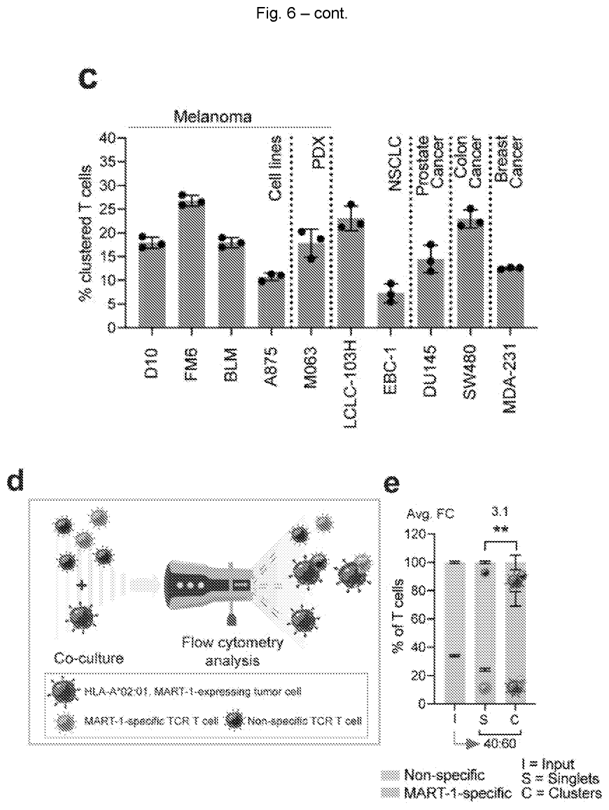

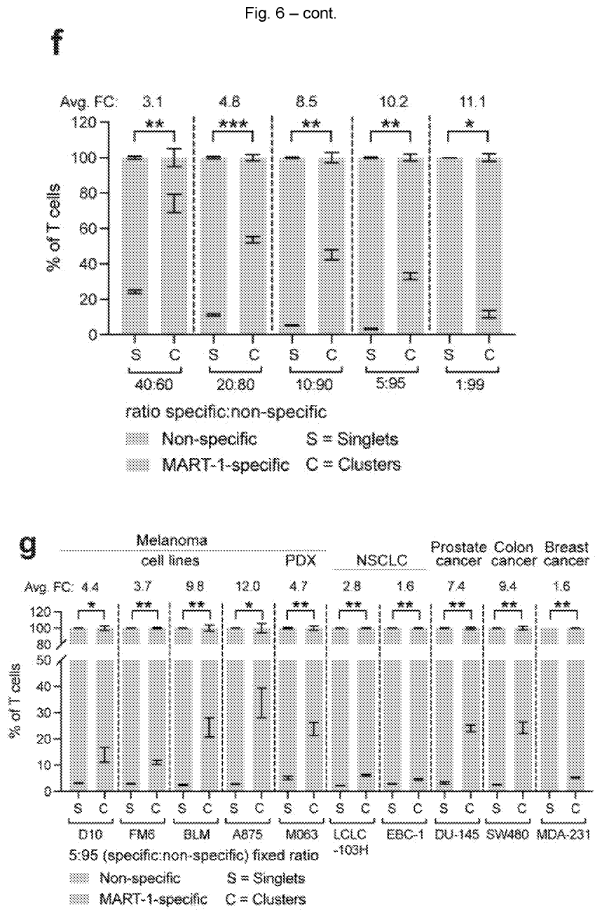

To study functional interactions between human T cells and tumor cells, we used a matched co-culture model established previously (Vredevoogd, 2019; Ibáñez-Molero, 2022). We engineered melanoma cells to express both HLA-A*02:01 and the MART-1 tumor antigen, as well as an mPlum fluorescent marker. CD8+ T cells were isolated from healthy donors, retrovirally transduced with a MART-1-specific TCR and labeled with cell trace violet (CTV). In a flow cytometry analysis performed after a 4 h co-culture, we observed single tumor cells and single T cells. Furthermore, we noticed a cell population that was positive for both the tumor and T cell markers, suggestive of the formation of heterotypic clusters (FIG. 6a). To validate the composition of this double-positive subpopulation, we used an imaging flow cytometer (Imagestream®). This analysis confirmed that at least 70% of this population comprised heterotypic cell:cell interactions (FIG. 6b). In most cases, this was accompanied by the formation of an immunological synapse, as judged by the relocalization of HLA-A*02 specifically to the T cell:tumor cell interface (FIG. 6b). To determine whether this was an observation peculiar to melanoma, we expanded it to other cancer cell lines. We observed that also for four other cancer indications, heterotypic T cell:tumor cell clusters formed after 4 h co-culture, reaching up to 26.7% of the T cell population (FIG. 6c).

This observation led us to investigate whether non-specific and antigen-specific T cells differentially engage with tumor cells to form conjugates. We admixed non-specific (appr. 60%) and MART-1-specific (appr. 40%) T cells to compete for association with tumor cells. After a 4 h co-culture, we evaluated the contribution of each T cell group to the clusters (FIG. 6d). We observed a>3-fold enrichment of MART-1-specific T cells over non-specific T cells in heterotypic tumor cell clusters (FIG. 6e). Next, we challenged the system to mimic a more physiologic setting, in which tumor-reactive T cells are underrepresented. We used a titration range of antigen-specific T cells in a co-culture with tumor cells. For all titrations, MART-1-specific T cells outcompeted their non-specific counterparts for cluster formation. Even when specific T cells accounted for only 1% of all T cells, they were enriched in tumor cell clusters (FIG. 6f). Again, this competitive advantage of antigen-specific T cells was not limited to melanoma, but reproduced across different cancer indications (FIG. 6g). In all cases, tumor cell conjugation led to increased activation of antigen-specific T cells, as judged by CD69 induction (Data not shown). To test the robustness of the system, we also inverted these titrations: when 95% of MART-1-specific T cells were mixed with 5% of non-specific T cells, we observed a depletion of the latter from tumor cell clusters. Together, these results indicate that in defined co-cultures, T cells and tumor cells form heterotypic clusters, in which antigen-recognizing T cells outcompete non-specific T cells.

Heterotypic clusters of CD8 T cells with tumor and/or APCs from clinical melanoma specimens

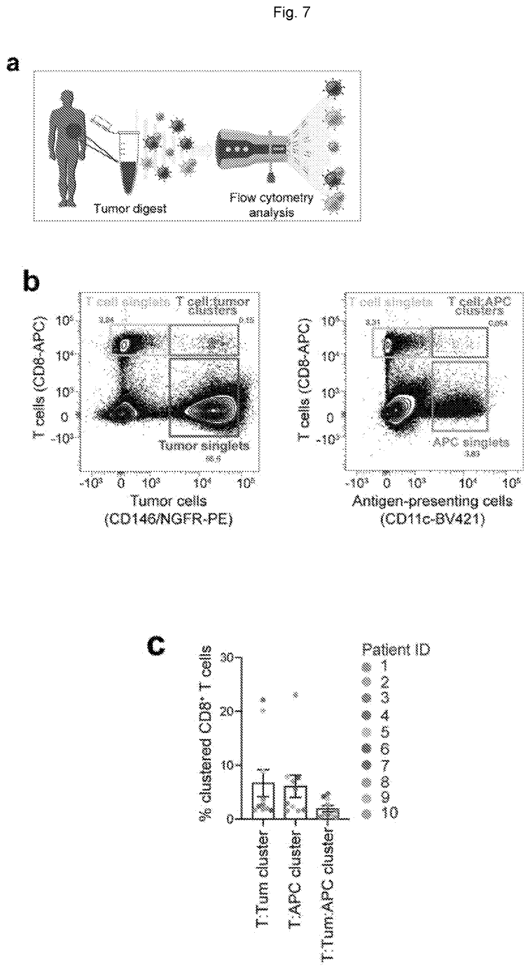

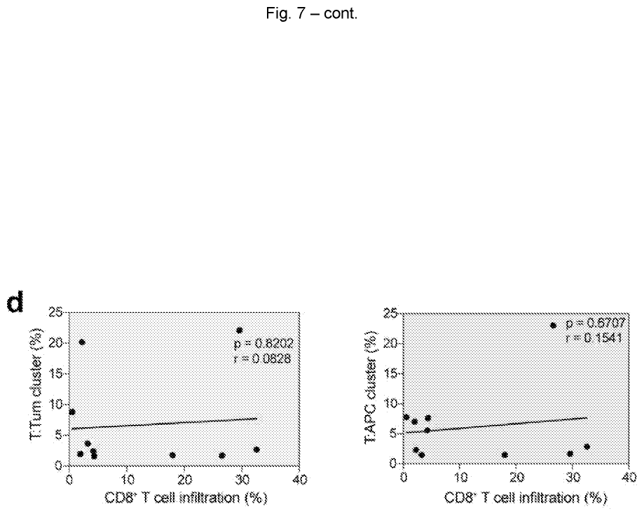

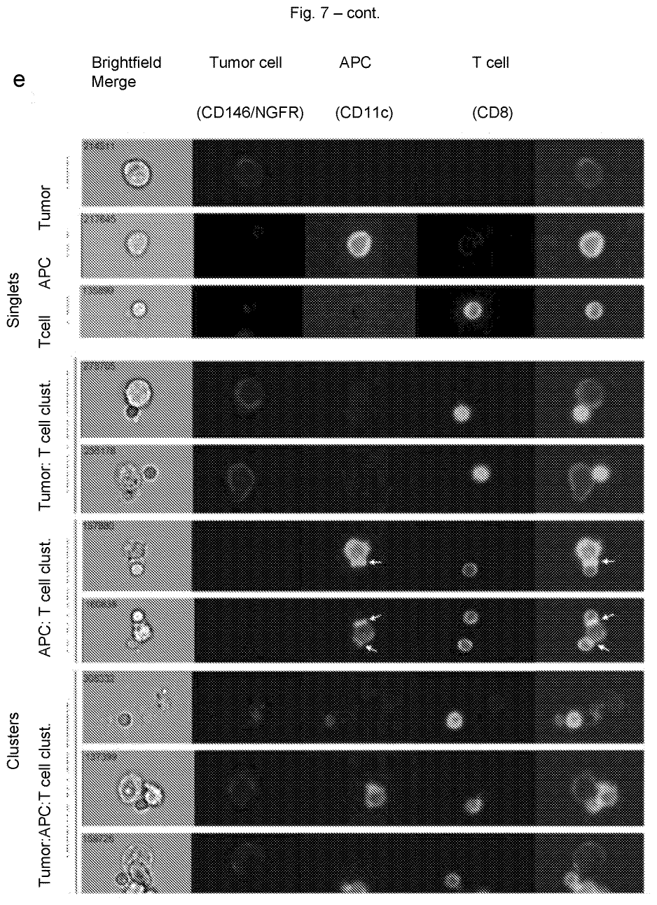

These observations, together with the reported correlations between CD8+ T cell-tumor cell proximity and immunotherapy response, prompted us to investigate whether heterotypic clusters between CD8+ T cells and tumor cells can be also isolated from clinical cancer specimens. We analyzed a cohort of ten melanoma metastases, eight in lymph nodes and two in-transit. Upon surgical removal the tissue was cut into small fragments and digested for 30 min in the presence of apoptosis inhibitors. Then, the samples were analyzed by flow cytometry using antibodies for CD146 and NGFR for melanoma cells and CD8+ for T cells. Because of the prevalence of APCs especially in lymph nodes, we also included a CD11c marker (FIG. 7a). As expected, we identified single cells (“singlets”) for each of CD8+ T cells, melanoma cells and APCs. More importantly, we also isolated heterotypic CD8+ T cell:melanoma cell clusters and CD8+ T cell:APC clusters, for 10/10 patient samples (FIGS. 7b and c). In addition to dual clusters, we observed clusters that were positive for T cell, tumor cell and APCs markers, suggestive of triple cell conjugates (FIG. 7c). The number of clusters did not correlate with the degree of T cell infiltration into tumors (FIG. 7d).

To validate the flow cytometry data and determine the composition of the cell clusters, we performed Imagestream® analysis on these clinical specimens. We observed single cells for all three cell types. Furthermore, we detected heterotypic cell clusters, comprising one or more T cells conjugated to either one or more tumor cells and/or one or more APCs (FIG. 7e). For approximately 61% of T cell:APC clusters an immunological synapse was seen, with CD11c relocalized to this specific interface. These results confirm that heterotypic CD8+ T cell clusters can be detected in, and isolated from, clinical cancer specimens.

CD8+ T cells from clusters display tumor-reactive cell states and increased TCR clonality

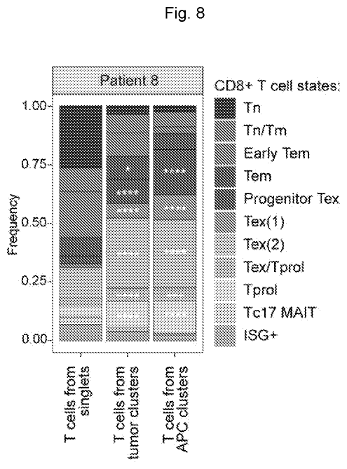

Next, it was important to characterize the clustered CD8+ T cells. We determined both their cell state and TCR clonality using single-cell (sc) RNA and TCR sequencing, corresponding to two key characteristics of T cell activity. Similar to FIG. 7, melanoma specimens were digested briefly and FACSorted to obtain CD8+ T cell, tumor cell and APC singlets and clusters. The sorting caused most clusters to dissociate into T cell, tumor cell and APCs singlets, which were captured into Gel Bead in Emulsion (GEM) droplets and subjected to sequencing. UMAP visualization of scRNA-seq data from all sequenced cells of all patients combined identified three distinct clusters: T cells, tumor cells and APCs, as expected. We then separated and computationally re-clustered all CD8+ T cell populations (derived from both singlets and clusters) to annotate them for their respective cell states based on RNA expression of T cell related genes and cross-labelling with external single-cell datasets of human CD8+ TILs20,21. We identified 11 cell states, all of which have been described previously20,21 (FIG. 8). Of note, this included a population of proliferating T cells (Tprol), which was marked not only by very high expression of proliferation genes but also showed exhaustion markers.

To compare cell states of T cells derived from singlets and clusters, we analyzed them individually. T cell singlets were enriched for naive and (early) effector memory T cell signatures. In contrast, T cells derived from both tumor and APC clusters showed a more exhausted signature (progenitor Tex, Tex, Tex/Tprol, Tprol), including higher expression of TNFRSF9, PDCD1, CXCL13, TIGIT, CTLA4, TOX (FIG. 8). To evaluate whether these cells were associated with a tumor-reactive phenotype, we assessed the CD8 neoantigen-specific TCR (neoTCR8) gene signature (Lowery et al., 2022). T cells from clusters showed increased expression of this signature compared to those from singlets while neoTCR8-high cells corresponded to those associated with exhausted T cell states (Data not shown). As an additional measure of tumor reactivity, we plotted the top-15 most frequent TCR clonotypes (combined unique CDR3a and β sequences). This revealed that relative to T cell singlets, T cells from tumor clusters (2/4 patients) and APC clusters (4/4 patients) were enriched for clonal TCRs (Data not shown). Together, these data indicate that CD8+ T cells from clusters, compared to T cell singlets, show a more exhausted phenotype, a higher neoTCR8 score and increased TCR clonality.

Expanded CD8+ T cells from clusters show increased cytokine production and killing of autologous tumor cells

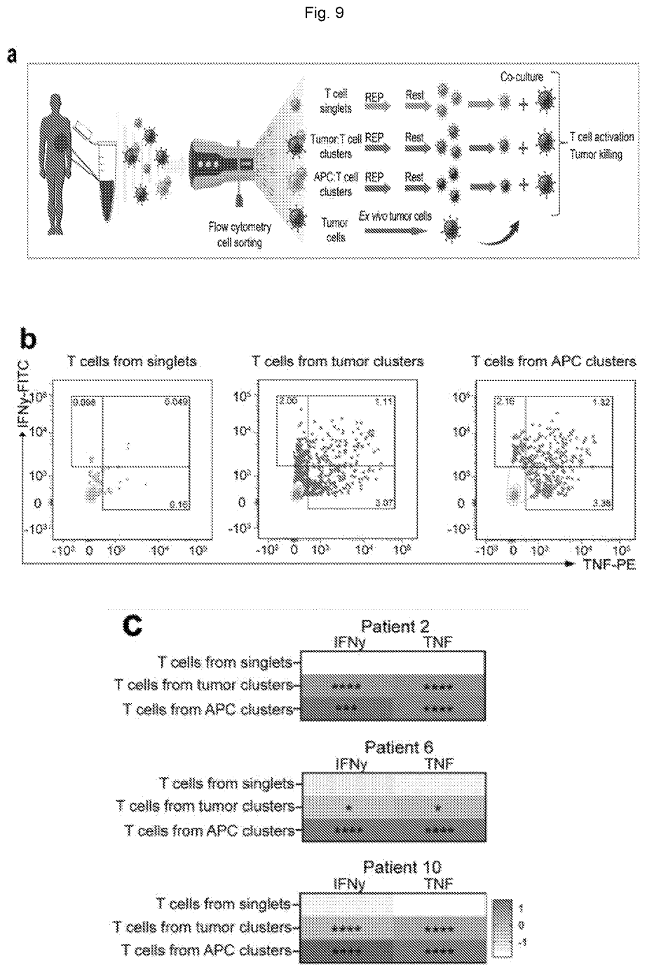

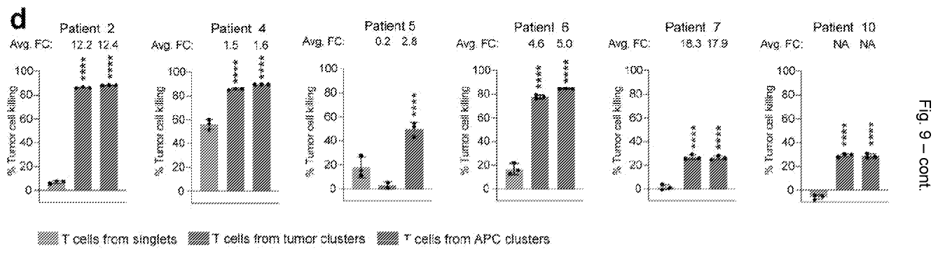

These results led us to investigate whether these three characteristics of clustered CD8+ T cells translate into improved tumor cell reactivity. We again isolated T cell singlets and clusters from melanoma digests by FACSorting, but this time this was followed by a Rapid Expansion Protocol (REP). T cells were allowed to rest and subsequently used to treat autologous melanoma cells ex vivo. This was followed by two types of analysis: T cell activation and tumor killing assays (FIG. 9a).

After a 4 h exposure to tumor cells, production of the cytokines IFNy and TNF was increased for T cells from clusters compared to T cells from singlets, indicative of higher activation (FIGS. 9b and c). Then, we determined the tumor reactivity of T cells from singlets and clusters. Autologous melanoma cells were established from the clinical samples and co-cultured with T cells for several days. As a measure of cell killing, we performed CellTiter-Blue assays, using untreated tumor cells as controls. Killing capacity of CD8+ T cells derived from tumor clusters was increased for 5/6 patients compared to T cell singlets, while for T cells from APCs clusters this was seen in 6/6 patients. Relative to T cell singlets, T cell-tumor cell clusters showed 7.4-fold higher activity while T cell-APC clusters were 7.9-fold more active. We conclude from this analysis that CD8+ T cells derived from cell clusters exert greater antitumor activity than single T cells (FIG. 9d).

To study the TME, much effort has focused on the preparation of single cells, which has significantly advanced our understanding of its composition and complexity. However, there are increasing indications that cells can engage in stable homo- and heterotypic interactions that are associated with distinct phenotypes and activities. The results presented here argue that for CD8+ T cells interacting with cancer cells and APCs, critical additional information and functionality can be unlocked when they are isolated from cell clusters instead of single cells. We demonstrate that these heterotypic CD8+ T cell clusters can be retrieved from clinical cancer samples, and more importantly, that T cells derived from these cellular conjugates are functionally distinct in that they markedly outperform single T cells in exerting antitumor activity. Our results extend previous observations on cellular conjugates, for example CTC clusters with different properties26,34-36, and interacting CD4+ T-APCs enriched in the TME and analyzed by PIC-seq27,28. We used imaging flow cytometry to validate the cell clusters, together with single cell RNA profiling of cluster-derived cells and functional assays. The output of these analyses is that CD8+ T cells derived from both tumor cell and APCs clusters are associated with (i) an exhausted phenotype, (ii) increased TCR clonality, (iii) augmented cytokine production and (iv) superior tumor cell elimination.

Our findings align with the established knowledge that CD8+ T cells engage in physical interactions with their target cells, which are mediated by multiple molecular contacts within the immunological synapse, predominantly between the TCR and a specific antigenic peptide presented by MHC-114. All clinical samples we analyzed were derived from tumor digests that had undergone a freeze-thaw cycle prior to imaging, FACS and functional analyses. This suggests that the clusters were sufficiently stable to withstand a freeze-thaw cycle, but we cannot exclude that at least some interactions derived from dissociations and (re-) associations. Furthermore, all of the clinical samples were taken from melanoma metastases. Whereas most were derived from lymph nodes, two corresponded to in-transit metastases, illustrating that both CD8+ T cell-tumor cell and CD8+ T cell-APC clusters could be isolated from at least two distinct anatomical sites.

We demonstrate that CD8+ T cells from clusters are not only functionally distinct but reside also in different cell states. These profiles are typically seen for tumor-reactive T cells associated with exhausted states. Most of these studies focused on the identification of tumor-reactive T cells by using cell surface markers, including CD39 and PD-1. The procedure we present here is different, in that independent of these markers, we identify and isolate a subpopulation of CD8+ T cells displaying high tumor reactivity. More importantly, this represents a group of highly valuable cells that is excluded from common cell sorting procedures.

Recently, the interaction of CD8+ T cells with APCs in the TME has gained considerable attention. It was shown that formation of type 1 conventional dendritic cells (cDC1)-CD8+ clusters at the tumor-stromal interface is associated with protective cancer immunity (Meiser et al, 2023). In line with this, the presence of stem-immunity hubs with CD8+ T cells adjacent to CXCL10+ macrophages in human lung cancers was found beneficial for response to PD-1 blockade. However, T cells can also be ‘trapped’ by these interactions to reduce their access to tumor cells, thereby limiting treatment efficacy. The interactions of CD8+ T cells with macrophages or DCs can be long-lasting antigen-specific synaptic interactions. However, this can prime tumor-specific T cells for either activation or exhaustion, which largely depends on the interacting APC type (Meiser et al, 2023). We show here that particularly CD8+ T cells conjugated to APCs are distinct in cell state, clonality and functionality, even more so than T cells from tumor cell clusters, emphasizing the importance of this population.

Lastly, considering the distinct properties and activities of CD8+ T cells from clusters compared to single T cells, our results suggest that these clusters harbor biologically relevant information that can now be further mined. In addition, our findings merit preclinical exploration. For the functional assays to evaluate T cell killing capacity of autologous tumor digests, we used a REP protocol not too dissimilar from that used in a recent positive clinical TIL trial for advanced melanoma52. It is based on ex-vivo outgrowth and expansion of TILs from digested tumors and subsequent reinfusion of the expanded product into the patient. However, neither characterization nor functional validation for T cell tumor reactivity is included in this clinical protocol. The results presented here raise the possibility that cluster isolation may be used to enrich tumor-reactive T cells from patients' tumors, which can be explored in preclinical settings.

### Material and Methods of Example 2

### Cell Lines

Human cancer cell lines were obtained from the Peeper lab repository. They were STR profiled and mycoplasma tested at the start of in vitro experiments. They were trypsinized and splitted weekly to maintain them at sub-confluency. They were transduced with lentivirus to express HLA-A*02:01-MART1-mPlum plasmid as described previously30. D10, FM6, BLM, A875, M063 and MDA-231 were cultured in DMEM (41966052, Gibco) with 10% fetal bovine serum (FBS, 3101120, Sigma) and 1:100 of 10.000U/ml Penicillin-Streptomycin (P/S, 15140122, Invitrogen). LCLC-103H, EBC-1, Du-145 and SW-480 were cultured in RPMI (21875034, Thermofisher) with 10% FBS and 1:100 of 10.000 U/ml P/S. For REP experiments, the suspension cell line EBV-JY was used. This cell line was not transduced and cultured in IMDM (CA IMDM-A, Capricorn Scientific) supplemented with 10% FBS and 1:100 10.000 U/mL P/S.

### Primary Human CD8 T Cells Isolation, Transduction and Culture

Primary CD8+ T cells used in in vitro experiments with cell lines were isolated from blood of healthy donors (specifically, from buffy coats). Briefly, PBMCs were isolated by density centrifugation using Ficoll (11743219, Fisher Scientific) (2500 rpm, 15 min, no break). CD8+ T cells were positively isolated with dynabeads (11333D, Invitrogen) and activated for 48 h in a pre-coated plate with α-hCD3 and α-hCD28 (16-0037-85/16-0289-85, eBioscience), 5 mg per well in 24-well plates at 10⁶ cells/ml. Then, CD8+ T cells were lentivirally transduced in retronectin (T100B, Takara)-coated plates with the MART-1-specific TCR (2000 g, 1.5 h, no break). For the first two days after activation, primary CD8+ T cells were cultured in RPMI with 10% FBS and 1:100 of 10.000 U/ml P/S, with IL2, IL7 and IL15 (100 IU/mL, 10 ng/ml, 10 ng/ml respectively) (Proleukin, Novartis; 11340075, Immunotools; 11340155, Immunotools). After that T cells were refreshed three times a week with RPMI containing 10% FBS, 1:100 of 10000 U/mL P/S and 100 IU/mL IL2.

### Patient Samples

Resected tumor material was collected from melanoma patients undergoing surgery at the Netherlands Cancer Institute (NKI-AvL) (Data Table 3). The study was approved by the Medical Ethical Review Board of the NKI-AvL (under studies B16MEL, IRBm23-029) and executed in compliance with the ethical regulations. All patients provided prior informed consent to research usage of material not required for diagnostics.

TABLE 3
Patient characteristics of resection samples used

| Sample ID | Site | Type | Used for |
|---|---|---|---|
| Patient 1 | Lymph node | Metastasis | scRNA/TCR-seq |
| Patient 2 | Lymph node | Metastasis | REP, scRNA/TCR- |
| | | | seq |
| Patient 3 | Lymph node | Metastasis | REP |
| Patient 4 | Dermal/subcutaneous | In transit | REP |
| | | metastasis | |
| Patient 5 | Lymph node | Metastasis | REP |
| Patient 6 | Subcutaneous | In transit | REP |
| | | metastasis | |
| Patient 7 | Lymph node | Metastasis | REP |
| Patient 8 | Lymph node | Metastasis | REP, scRNA/TCR- |
| | | | seq |
| Patient 9 | Lymph node | Metastasis | scRNA/TCR-seq |
| Patient 10 | Lymph node | Metastasis | REP |
| Patient 11 | Lymph node | Metastasis | * |

### Patient Tumor Digestion

To achieve tumor digests, freshly obtained patient tumors were cut in small pieces and incubated in pre-warmed RPMI medium supplemented with pulmozyme (12.6 μg/mL) (Roche), collagenase (1 mg/mL) (17104-019, ThermoFisher Scientific) and a pan-caspase inhibitor (Q-VD-Oph 50 UM or Z-VAD 5 μg/mL) (S7311, Selleckchem; sc-3067, Santa Cruz Biotechnology) at 37 degrees in a spinning rotor for a maximum of 30 minutes. The sample was then passed through a 100 μm filter, washed with RPMI containing 10% FBS and frozen in FBS+10% DMSO until further processing.

### IN Vitro T Cell: Tumor Cell Line Co-Cultures

Before start of co-culture, primary CD8+ T cells were labeled with cell trace violet (CTV) (C34557, Invitrogen) or carboxyfluorescein succinimidyl ester (CFSE) (C34554, Invitrogen) according to manufacturer's instructions. Tumor cell lines and pre-labeled CD8+ T cells were counted and seeded in a non-tissue culture treated 96 well V-bottom plate (781601, Brand) at 2:1 tumor: T cell ratio for standard flow cytometry and 1:1 ratio for image based flow cytometry assays (50.000 tumor and 25.000/50.000 T cells, respectively). Co-culture was performed in 100 μl per well with 50 μl of tumor cell medium and 50 μl of T cell medium with IL2. In standard assays cells were co-cultured for 4 h and subsequently analyzed by flow cytometry. When required, multiple wells were pooled for analysis. For competition assays, non-specific and MART-1-specific T cells were mixed at the indicated ratios before the start of co-culture, based on the measured transduction efficiency. After most co-cultures, the percentage of MART-1-specific T cells in the populations of interest was determined by staining for the mouse TCR beta chain. For the experiment, in which the 5:95 and 95:5 ratios (MART-1-specific:non-specific) were studied together, the T cells were sorted after transduction to get a pure MART-1-specific T cell population. Before co-culture, MART-1-specific T cells were then stained with CTV and non-specific T cells with CFSE, after which they were mixed at the above ratios to perform the co-culture.

### Flow Cytometry and Cell Sorting

For flow cytometry, culture medium was removed and cells were washed with 0.1% BSA in PBS. For surface staining, cells were stained with the indicated antibodies diluted in 0.1% BSA in PBS for 30 min on ice in dark. For intracellular staining, cells were stained using the FOXP3 kit (00-5523-00, Invitrogen) according to manufacturer's instructions. A list of antibodies used can be found in Table 4. After staining, cells were washed twice with 0.1% BSA in PBS and analyzed using a BD LSRFortessa flow cytometer. For primary human tumor samples, previously frozen tumor digest was thawed and washed twice with RPMI, supplemented with 10% FBS and 1:1000 benzonase nuclease (purity>90%) (70746-3, VWR). Cells were washed an additional time with 0.1% BSA in PBS after which they were stained with antibody mix for 30 minutes on ice in the dark. After staining, cells were washed twice with 0.1% BSA in PBS before proceeding towards flow cytometry or sorting. When indicated, samples were washed and stained with 2% BSA in PBS and sorted in 2% FBS in PBS. Cell sorting was performed using a BD FACSAria Fusion cell sorter with a 80, 100 or 130 μM nozzle depending on the size of cells sorted. Sorted cells were collected in RPMI supplemented with 20% FBS, before proceeding to downstream processing.

TABLE 4
Reagents and antibodies used for flow cytometry staining

| | | | | Catolog | |
|---|---|---|---|---|---|
| Antibody | Dilution | Clone | Company | Number | Used for |
| PE Hamster Anti-Mouse TCRβ chain | 1:100 | H57- | BD | 553172 | In vitro - % MART-1 specific T |
| | | 597 | Biosciences | | cells |
| APC Hamster Anti-Mouse TCRβ chain | 1:100 | H57- | BD | 553174 | In vitro - % MART-1 specific T |
| | | 597 | Biosciences | | cells |
| APC Anti-human CD69 | 1:100 | FN50 | Biolegend | 310910 | In vitro - T cell activation |
| PE Anti-human CD69 | 1:100 | FN50 | Immunotools | 21620694X2 | In vitro - T cell activation |
| FITC Mouse Anti-human HLA-A2 | 1:100 | BB7.2 | BD | 551285 | In vitro - ImageStream |
| | | | Biosciences | | |
| PE Mouse Anti-human CD271 (NGFR) | 1:100 | C40- | BD | 557196 | Sort REP, ImageStream |
| | | 1457 | Biosciences | | |
| APC Anti-human CD271 (NGFR) | 1:100 | ME20.4 | Biolegend | 345108 | Sort sc-sequencing, sort REP, |
| | | | | | flow cytometry-cytokine |
| | | | | | production |
| PE Anti-human CD146 | 1:100 | P1H12 | Biolegend | 361006 | Sort REP, ImageStream |
| APC Anti-human CD146 | 1:100 | P1H12 | Biolegend | 361016 | Sort sc-sequencing, sort REP, |
| | | | | | flow cytometry-cytokine |
| | | | | | production |
| BV421 Anti-human CD11c | 1:40 | Bu15 | Biolegend | 337226 | Sort sc-sequencing, sort REP, |
| | | | | | ImageStream |
| BUV805 Mouse Anti-Human CD8 | 1:50 | SK1 | BD | 612889 | Sort sc-sequencing, sort REP |
| | | | Biosciences | | |
| PE Anti-human CD8 | 1:50 | SK1 | Biolegend | 344706 | Sort sc-sequencing, sort REP |
| APC Anti-human CD8a | 1:50 | RPA-T8 | Biolegend | 301049 | Sort REP, ImageStream |
| CD3 FITC | 1:50 | SK7 | BD | 345763 | Sort sc-sequencing |
| | | | Biosciences | | |
| FITC Mouse Anti-Human INF-γ | 1:100 | B27 | BD | 554700 | Flow cytometry - Cytokine |
| | | | Biosciences | | production |
| PE Anti-human TNF-α | 1:100 | Mab11 | Biolegend | 502909 | Flow cytometry - Cytokine |
| | | | | | production |
| TotalSeq-C0251 anti-human Hashtag 1 | 1:500 | LNH-94; | Biolegend | 394661 | Sc-sequencing - To label of |
| | | 2M2 | | | tumor: CD8 doublets |
| TotalSeq-C0252 anti-human Hashtag 2 | 1:500 | LNH-94; | Biolegend | 394663 | Sc-sequencing - To label |
| | | 2M2 | | | APC: CD8 doublets |
| LIVE/Dead Fixable Near-IR Dead Cell | 1:1000 | — | Invitrogen | L34876 | Flow cytometry/Sort - Live/dead |
| Stain Kit | | | | | |
| CellTrace Violet Cell Proliferation Kit | 1:4000 | — | Invitrogen | C34557 | In vitro and secondary co- |
| | | | | | cultures |
| CellTrace CFSE Cell Proliferation Kit | 1:4000 | — | Invitrogen | C34554 | In vitro co-cultures |

### Imagestream® Analysis

For Imagestream® analysis samples were processed following the flow cytometry staining procedure described above and diluted to 10×10⁶ cells/ml in 0.1% BSA in PBS after the final wash. Cells were analyzed using Imagestream® and obtained data was processed using IDEAS® software. Data was exported as individual OME tiff53 files. A custom image analysis workflow was developed in FIJI54. Cellpose was used for cell segmentation. This required a nuclear and membranous signal. The membranous signal was obtained by applying a variance filter of radius 2 on the brightfield image. For in vitro samples the nuclear signal was obtained by combining the normalized signal from the nuclear markers. For patient derived samples all fluorescence channels were added to the filtered brightfield channel after normalization. After segmentation the membrane was estimated by eroding the regions with 2 pixels and subsequently expanding by 3 pixels. The interface between cells could then be found by checking for overlap between regions. The intensity of the marker of interest in the interface and membrane regions were measured using CLIJ56.

### Single Cell and TCR Sequencing

Tumor digest was thawed, stained and sorted as described above. Five populations were sorted from live cells: tumor singlets (NGFR/CD146+); tumor: CD8+T cell clusters (NGFR/CD146+,CD8+); APC: CD8+ T cell clusters (NGFR−,CD146−, CD11c+, CD8+), CD8+ T cell singlets (NGFR−,CD146−,CD11c−,CD8+) and APC singlets (NGFR−,CD146−,CD11c+, CD8−). Singlets were pooled together during sort in a ratio of 1:1:1. If the amount of clusters was low, they were kept as separate samples. If sufficient amounts of clusters were sorted (>40.000 clusters), clusters were hashtagged and pooled 1:1 after the sort. For hashtagging, sorted cells were washed once with 2% BSA in PBS and incubated with the hashtagging antibody for 30 min on ice. After hashtagging, cells were washed an additional two times with 0.04% BSA in PBS, after which they were pooled. Cells that do not need hashtagging were washed twice with 0.04% BSA in PBS, before proceeding to single cell 5′ sequencing library preparation.

The Chromium Controller and Chromium X platform of 10× Genomics was used for single cell partitioning and barcoding. Each cell's transcriptome was barcoded during reverse transcription, pooled cDNA was amplified and Single Cell 5′ Gene Expression (GEX), V(D)J and Feature Barcode (FB) Libraries were prepared according to the manufacturer's protocol (CG000331, 10× Genomics). All libraries were quantified and normalized based on library QC data generated on the Bioanalyzer system according to manufacturer's protocols (G2938-90321 and G2938-90024, Agilent Technologies). Based on the expected target cell counts, a balanced library sub-pool of samples was composed for SC5′GEX, V(D)J and FB libraries. Library sub-pools were quantified by qPCR, according to the KAPA Library Quantification Kit Illumina® Platforms protocol (KR0405, KAPA Biosystems). Based on qPCR results a final sequencing pool was composed. Paired end sequencing was performed on a NovaSeq 6000 Instrument (Illumina) using NovaSeq 6000 Reagent Kits v1.5 100 cycles (cat. no. 20028401, 20028319, 20028316 Illumina), using 28 cycles for Read 1, 10 cycles for Read i7, 10 cycles for Read i5 and 90 cycles for Read 2.

Rapid Expansion Protocol (REP) of TILs from Patient Material

Tumor digest was thawed, stained and sorted as described above. Four populations were sorted from live cells: tumor singlets, tumor: CD8+ T cell clusters, APC: CD8+ T cell clusters and CD8+ T cell singlets. The REP was performed according to a protocol adjusted from Hombrink et a57I. Briefly, sorted CD8+ T cell populations were plated at 100-150 cells/well in round-bottom tissue culture treated 96-well plates (650-180, Greiner) in 100 μL RPMI medium supplemented with 10% human serum (H3667, Sigma-Aldrich), 5% FBS, 300 IU/mL IL-2, 10 ng/ml IL-7, 10 ng/ML IL-15, 0.8 μg/mL phytohaemagglutinin (PHA, R30852801, ThermoFisher Scientific) and 50.000 irradiated feeder cells. Feeder cells consisted of 45.000 35 Gray irradiated allogeneic PBMCs (mix of 2 donors) and 5000 50 Gy irradiated EBV-JY cell line. After 10-11 days, T cells were collected and rested for at least 3 days in RPMI medium supplemented with 10% FBS and 100 IU/mL IL-2, before functional tests were performed. Sorted melanoma tumor cells were cultured in tissue culture treated flat bottom plates in DMEM or Ham's F-10 medium (11550043, Gibco) supplemented with 10% FBS and 1:100 of 10.000 U/ml P/S and adherent cells were split when reaching confluency.

Secondary Co-Cultures after REP

Details on secondary co-cultures can be found in Table 5 (A and B). To assess cytokine production, CTV labeled CD8+ T cells were co-cultured with autologous melanoma tumor cells for 4 h in a ratio 1:1, after 2 h 1:1000 diluted Golgiplug (555029, BD) was added to the culture. After co-culture, an intracellular staining protocol was performed as described above and cytokine production was measured by flow cytometry. For killing assays, melanoma tumor cells were adhered to a tissue culture treated 96-well flat bottom plate, after which unlabelled T cells were added at the indicated ratios. At the end of co-culture, T cells were removed from the plate and tumor cell viability was determined using CellTiter-Blue (G8081, Promega) following manufacturers instructions.

TABLE 5
(A) Details on experiments with REP T cells Killing assay

| | | | Killing assay with autologus tumor cells | | | | | | | |
|---|---|---|---|---|---|---|---|---|---|---|
| Sample | Sort | Performed | Performed | | | | | | | |
| ID | reagents | REP | experiment | Ratio | T cells | Tumor | Days | Restimulation | T cells | Days |
| Patient 1 | 0.1% BSA | No* | No* | | | | | | | |
| Patient 2 | 2% BSA, | Yes | Yes | 1:2; | 5000; | 10.000 | 2 days | No | | |
| | 2% FBS | | | 1:1; | 10.000; | | | | | |
| | | | | 2:1 | 20.000 | | | | | |
| Patient 3 | 0.1% BSA | Yes | No** | | | | | | | |
| Patient 4 | 2% BSA, | Yes | Yes | 1:8; | 7500; | 60.000 | 3 days | No | | |
| | 2% FBS | | | 1:4; | 15.000; | | | | | |
| | | | | 1:2 | 30.000 | | | | | |
| Patient 5 | 0.1% BSA | Yes | Yes | 1:2; | 5000; | 10.000 | 3 days | Yes | 15.000; | 4 days |
| | | | | 1:1 | 10.000 | | | | 45.000 | |
| Patient 6 | 2% BSA, | Yes | Yes | 1:4; | 5000; | 20.000 | 3 days | No | | |
| | 2% FBS | | | 1:2; | 10.000; | | | | | |
| | | | | 1:1 | 20.000 | | | | | |
| Patient 7 | 2% BSA, | Yes | Yes | 1:4; | 2500; | 10.000 | 1 day | Yes | 25.000; | 2 days |
| | 2% FBS | | | 1:2; | 5000; | | | | 50.000; | |
| | | | | 1:1 | 10.000 | | | | 100.000 | |
| Patient 8 | 2% BSA, | Yes | No** | | | | | | | |
| | 2% FBS | | | | | | | | | |
| Patient 9 | 2% BSA, | No | No | | | | | | | |
| | 2% FBS | | | | | | | | | |
| Patient 10 | 2% BSA, | Yes | Yes | 1:2; | 5000; | 10.000 | 2 days | No | | |
| | 2% FBS | | | 1:1 | 10.000 | | | | | |
| Patient 11 | — | No* | No* | | | | | | | |

(B) Details on experiments with REP T cells IFNyTNFa
production with autologous tumor cells

| | | IFNyTNFa production with autologous tumor cells | | | | |
|---|---|---|---|---|---|---|
| Sample ID | Sort reagents | Performed experiment | Ratio | Tumor | T cells | Time |
| Patient 1 | 0.1% BSA | No | | | | |
| Patient 2 | 2% BSA, 2% FBS | Yes | 1:1 | 50.000 | 50.000 | 4 h |
| Patient 3 | 0.1% BSA | No | | | | |
| Patient 4 | 2% BSA, 2% FBS | No | | | | |
| Patient 5 | 0.1% BSA | No | | | | |
| Patient 6 | 2% BSA, 2% FBS | Yes | 1:1 | 50.000 | 50.000 | 4 h |
| Patient 7 | 2% BSA, 2% FBS | No | | | | |
| Patient 8 | 2% BSA, 2% FBS | No | | | | |
| Patient 9 | 2% BSA, 2% FBS | No | | | | |
| Patient 10 | 2% BSA, 2% FBS | Yes | 1:1 | 50.000 | 50.000 | 4 h |
| Patient 11 | — | No | | | | |

* Not enough material
** Autologous tumor cells were not viable after sort

### Statistical Analysis

Throughout this and other examples, different statistical tests have been used as indicated in each figure legend. T-test was performed when comparing two conditions, 2-way ANOVA was performed when comparing more than two conditions and Pearson was performed for correlation analysis. Bioinformatics test will be added here. Investigators were not blinded to allocation during experiments and outcome assessment. Analysis were performed using Graphpad and R.

### Example 3

### CD8 and CD4 T Cell Clusters in Blood and Pleural Effusion Samples

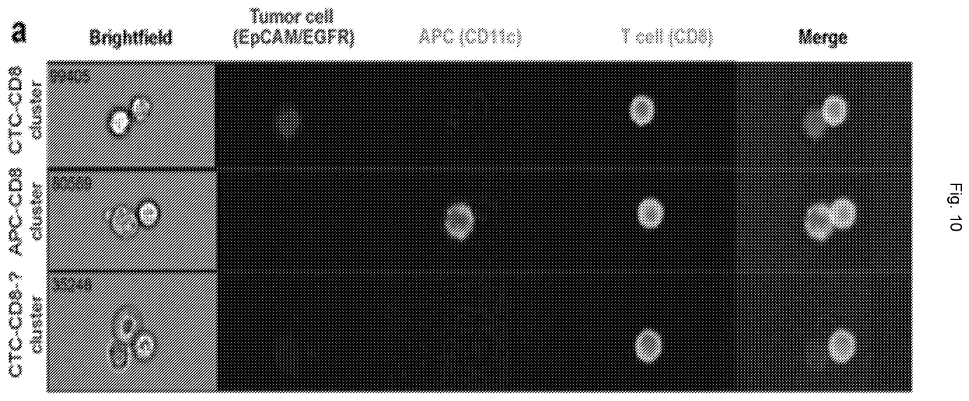

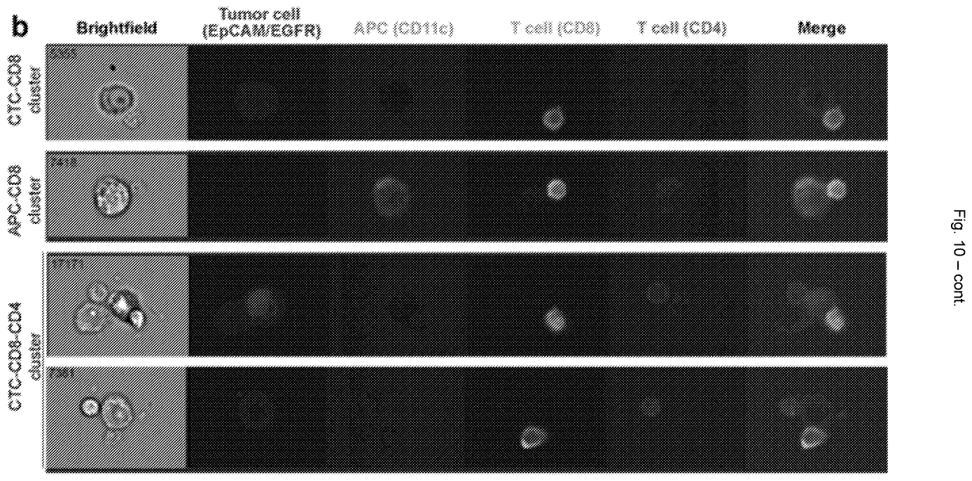

Following the same experimental setup as illustrated in previous examples, the inventors also determined the presence of T cells in cell:cell complexes in blood and pleural effusions from patients with tumors. Data are depicted in FIG. 10, wherein Cytotoxic T cells (CTC) and APCs in clusters with T cells (CD4 and CD8) in blood (a, top) and pleural effusion samples (b,bottom) were visualized by Imagestream®, following the previously disclosed methodology.

This data demonstrate that the cells of interest can be isolated from several kinds of samples.

Having now fully described this invention, it will be appreciated by those skilled in the art that the same can be performed within a wide range of equivalent parameters, concentrations, and conditions without departing from the spirit and scope of the invention and without undue experimentation.

Reference to known method steps, conventional methods steps, known methods or conventional methods is not in any way an admission that any aspect, description, or embodiment of the present invention is disclosed, taught, or suggested in the relevant art.

### REFERENCES

Anna Purcarea 1, Sebastian Jarosch 1, Jack Barton 1, Simon Grassmann 1, Ludwig Pachmayr 1, Elvira D'Ippolito 1, Monika Hammel 1, Anna Hochholzer 1, Karolin I Wagner 1, Joost H van den Berg 2, Veit R Buchholz 1, John B A G Haanen 3, Dirk H Busch 1 4 5, Kil, K. S. 1. (2022). Signatures of recent activation identify a circulating T cell compartment containing tumor-specific antigen receptors with high avidity. Science Immunology, 7, 74. https://doi.org/10.1126/sciimmunol.abm2077
Arnaud, M., Chiffelle, J., Genolet, R., Navarro Rodrigo, B., Perez, M. A. S., Huber, F., Magnin, M., Nguyen-Ngoc, T., Guillaume, P., Baumgaertner, P., Chong, C., Stevenson, B. J., Gfeller, D., Irving, M., Speiser, D. E., Schmidt, J., Zoete, V., Kandalaft, L. E., Bassani-Sternberg, M., . . . . Harari, A. (2022). Sensitive identification of neoantigens and cognate TCRs in human solid tumors. Nature Biotechnology. https://doi.org/10.1038/s41587-021-01072-6
Gomez-Eerland, R., Nuijen, B., Heemskerk, B., van Rooij, N., van den Berg, J. H., Beijnen, J. H., Uckert, W., Kvistborg, P., Schumacher, T. N., Haanen, J. B. a G., & Jorritsma, A. (2014). Manufacture of gene-modified human T-cells with a memory stem/central memory phenotype. Human Gene Therapy Methods, 25(5), 277-287. https://doi.org/10.1089/hgtb.2014.004
Kalbasi, A., & Ribas, A. (2020). Tumour-intrinsic resistance to immune checkpoint blockade. In Nature Reviews Immunology. https://doi.org/10.1038/s41577-019-0218-4
Kato, T., Matsuda, T., Ikeda, Y., Park, J. H., Leisegang, M., Yoshimura, S., Hikichi, T., Harada, M., Zewde, M., Sato, S., Hasegawa, K., Kiyotani, K., & Nakamura, Y. (2018). Effective screening of T cells recognizing neoantigens and construction of T-cell receptor-engineered T cells. Oncotarget. https://doi.org/10.18632/oncotarget.24232
Lu, Y. C., Zheng, Z., Lowery, F. J., Gartner, J. J., Prickett, T. D., Robbins, P. F., & Rosenberg, S. A. (2021). Direct identification of neoantigen-specific TCRs from tumor specimens by high-throughput single-cell sequencing. Journal for Immuno Therapy of Cancer. https://doi.org/10.1136/jitc-2021-002595
Rapoport, A. P., Stadtmauer, E. A., Binder-Scholl, G. K., Goloubeva, O., Vogl, D. T., Lacey, S. F., Badros, A. Z., Garfall, A., Weiss, B., Finklestein, J., Kulikovskaya, I., Sinha, S. K., Kronsberg, S., Gupta, M., Bond, S., Melchiori, L., Brewer, J. E., Bennett, A. D., Gerry, A. B., . . . . June, C. H. (2015). NY-ESO-1-specific TCR-engineered T cells mediate sustained antigen-specific antitumor effects in myeloma. Nature Medicine. https://doi.org/10.1038/nm.3910
Rosenberg, S. A., Spiess, P., & Lafreniere, R. (1986). A new approach to the adoptive immunotherapy of cancer with tumor-infiltrating lymphocytes. Science. https://doi.org/10.1126/science.3489291
Sharma, P., Hu-Lieskovan, S., Wargo, J. A., & Ribas, A. (2017). Primary, Adaptive, and Acquired Resistance to Cancer Immunotherapy. Cell, 168(4), 707-723. https://doi.org/10.1016/j.cell.2017.01.017
Van Den Berg, J. H., Heemskerk, B., Van Rooij, N., Gomez-Eerland, R., Michels, S., Van Zon, M., De Boer, R., Bakker, N. A. M., Jorritsma-Smit, A., Van Buuren, M. M., Kvistborg, P., Spits, H., Schotte, R., Mallo, H., Karger, M., Van Der Hage, J. A., Wouters, M. W. J. M., Pronk, L. M., Geukes Foppen, M. H., . . . . Haanen, J. B. A. G. (2020). Tumor infiltrating lymphocytes (TIL) therapy in metastatic melanoma: Boosting of neoantigen-specific T cell reactivity and long-term follow-up. Journal for Immuno Therapy of Cancer. https://doi.org/10.1136/jitc-2020-000848
Voabil, P., de Bruijn, M., Roelofsen, L. M., Hendriks, S. H., Brokamp, S., van den Braber, M., Broeks, A., Sanders, J., Herzig, P., Zippelius, A., Blank, C. U., Hartemink, K. J., Monkhorst, K., Haanen, J. B. A. G., Schumacher, T. N., & Thommen, D. S. (2021). An ex vivo tumor fragment platform to dissect response to PD-1 blockade in cancer. Nature Medicine. https://doi.org/10.1038/s41591-021-01398-3
Zheng, L. et al. Pan-cancer single-cell landscape of tumor-infiltrating T cells. Science 374, abe6474 (2021).
Oliveira, G. et al. Phenotype, specificity and avidity of antitumour CD8+ T cells in melanoma. Nature (2021) doi: 10.1038/s41586-021-03704-y.
Lowery, F. J. et al. Molecular signatures of antitumor neoantigen-reactive T cells from metastatic human cancers. Science (2022) doi: 10.1126/science.abl5447.
Vredevoogd, D. W. et al. Augmenting Immunotherapy Impact by Lowering Tumor TNF Cytotoxicity Threshold. Cell 178, 585-599.e15 (2019).
Ibáñez-Molero, S. et al. SERPINB9 is commonly amplified and high expression in cancer cells correlates with poor immune checkpoint blockade response. Oncolmmunology 11, 2139074 (2022).
Meiser, P. et al. A distinct stimulatory cDC1 subpopulation amplifies CD8+ T cell responses in tumors for protective anti-cancer immunity. Cancer Cell S1535610823002180 (2023) doi: 10.1016/j.ccell.2023.06.008.

1. A method for isolating T cells from a sample obtained from a subject, wherein the subject is diagnosed with cancer and/or is suspected of having cancer, the method comprising at least the steps of:
(i) obtaining or providing a sample from the subject, wherein the sample is
(a) a tumor tissue sample; and/or
(b) a blood sample, and/or
(c) lymph node sample
(ii) treating the sample by:
(a) in case of a tumor tissue sample and/or a lymph node sample, dissociating the tumor tissue sample and/or lymph node sample; and/or
(b) in case of a blood sample, lysing or removing erythrocytes from the blood sample, and
(iii) identifying in and/or isolating from the sample obtained after step (ii) a cell-cell complex comprising a T cell, and/or a T cell comprised in the cell-cell complex.

2. The method of claim 1, wherein the cell-cell complex is a T cell-tumor cell complex and/or wherein the cell-cell complex is a T cell-antigen-presenting cell (APC) complex, preferably wherein the APC is a dendritic cell (DC), a B-cell and/or a macrophage, and/or any other antigen-presenting cell.

3. The method of claim 1, wherein the method further comprises identifying and/or isolating a tumor cell and/or an APC comprised in the cell-cell complex.

4. The method of claim 1, wherein the cell-cell complex comprises more than one T cell and/or more than one tumor cell and/or more than one APC.

5. The method of claim 1, wherein the ratio in the cell-cell complex of T cell:non-T cell is between 1:10 and 10:1, preferably between 1:5 and 5:1, even more preferably between 1:2 and 2:1, most preferably 1:1.

6. The method of claim 1, wherein the T cells are tumor-reactive cytotoxic T cells.

7. The method of claim 1, wherein the method comprises freezing the sample after step (ii), followed by thawing of the sample before step (iii), or wherein the sample is a sample that is not frozen.

8. The method of claim 1, wherein the method comprises treating the sample with a cell death inhibitor, preferably an apoptosis inhibitor, preferably wherein the apoptosis inhibitor is a caspase inhibitor, preferably selected from ZVAD (zVAD-fmk), VAD, YVAD, zYVAD-fmk, XIAP, BIR, cIAP-1, cIAP-2, or Quinoline-Val-Asp-Ch2-O-Ph (QVD), preferably wherein the contacting is at least in step (i) and/or (ii).

9. The method of claim 1, wherein the method comprises performing a disaggregation step on the sample before, during or after step (ii).

10. The method of claim 1, wherein dissociating the tumor tissue sample and/or lymph node sample in step (ii) comprises enzymatic treatment of the tumor tissue sample and/or the lymph node sample to dissociate the cells in the tissue, preferably wherein at least a DNAase and/or a collagenase is used, and/or wherein a cell death inhibitor is used, and, preferably, wherein the treatment is for a period of between 5-60 minutes, for example 10-40 minutes or 25-35 minutes, at a temperature between 30-40 degrees Celsius, for example 36-38 degrees Celsius.

11. The method of claim 1, wherein lysing or removing erythrocytes from the blood sample in step (ii) comprises treatment of the blood sample with a Red Blood Cell Lysing Buffer, preferably for a period of 5-25 minutes, for example 10-15 minutes, at a temperature of between 15-25 degrees Celsius.

12. The method of claim 1, wherein in step (iii) the cell-cell complex comprising a T cell is identified and/or isolated by using a T cell-specific marker.

13. The method of claim 1, wherein in step (iii) the cell-cell complex comprising a T cell is identified and/or isolated by using a T cell-specific marker and a tumor cell-specific marker, and/or by using a T cell-specific marker and an APC-specific marker, and/or by using a T cell-specific marker, a tumor-specific marker and an APC-specific marker, and preferably wherein the cell-cell complex comprising a T cell is further identified and/or isolated by determining T cell activation of the T cell comprised in the cell-cell complex, preferably using a T cell activation specific maker, preferably wherein the T cell activation specific maker is CD69, or any other marker, and/or genetic signature, that is associated with T cell activity.

14. The method of claim 1, wherein the method further comprises determining T cell activation of the T cell comprised in the cell-cell complex, preferably wherein T cell activation is determined using CD69 as a marker, or any other marker, and/or genetic signature, that is associated with T cell activity and/or wherein the method further comprises identifying dead or dying tumor cells comprised in the cell-cell complex, preferably wherein dead or dying tumor cells are identified using a cell death marker, a dying cell marker, and/or a apoptosis marker, for example Annexin V and/or genetic signature that is associated with a dead or dying tumor cell, preferably in combination with one or more tumor-specific markers to identify the tumor cell.

15. The method of claim 1, wherein the T cell is a CD8+ T cell and/or a CD4+ T cell.

16. The method of claim 1, wherein the cell-cell complexes comprising a T cell are identified and/or isolated using cell sorting, flow cytometry, and/or imaging flow cytometry.

17. The method of claim 1, wherein the method further comprises identifying the T cell receptor expressed in the isolated T cell.

18. The method of claim 1, wherein the method further comprises in vitro expansion of the isolated T cell, APC and/or tumor cell.

19. A method of identifying a TCR useful in the treatment of a subject, a T cell useful in the treatment of a subject and/or an APC useful in the treatment of a subject and/or a tumor cell, the method comprising performing the method according to any one of the claims 1-18 to identify in step (iii) from the sample obtained after step (ii) the cell-cell complexes comprising a T cell, and wherein the method further comprises identifying/and or isolating the T cell and/or the APC and/or the tumor cell from the cell-cell complex, and/or identifying the TCR expressed in the isolated T cell, and preferably, wherein the identified and/or isolated T cell is expanded and used in adoptive T cell therapy (ACT), for example but not limited to TIL therapy.

20-22. (canceled)

23. A method of identifying a cell useful in the treatment of a subject, preferably a method of identifying a TCR useful in the treatment of a subject, a T cell useful in the treatment of a subject and/or an APC useful in the treatment of a subject, the method comprising performing the method according to any one of the claims 1-18 to identify and/or isolate in step (iii) from the sample obtained after step (ii) the cell-cell complexes comprising a T cell,
wherein the method further comprises:
identifying and/or isolating a cell from the cell-cell complex, preferably identifying/and or isolating a T cell and/or a APC from the cell-cell complex;
performing one or more of a gene expression analysis, and an omics analysis, preferably proteomic analysis, of the identified and/or isolated cell from the cell-cell complex, preferably of the T cell and/or of the APC identified/and or isolated from the cell-cell complex;
comparing the results of one or more of a gene expression analysis, and an omics analysis, with the results of the gene expression analysis and/or omics analysis performed on a cell, preferably on a T cell and/or on an APC, of the isolated sample obtained after step (ii), wherein said cell, preferably said T cell and/or APC, is not comprised in a cell-cell complex,
wherein one or more of a differentially expressed gene or differential omics is used as marker to identify a cell, preferably a T cell and/or an APC, to be expanded and/or a cell, preferably a T cell useful in the treatment of a subject and/or preferably an APC useful in the treatment of a subject, and/or TCR useful in the treatment of a subject.

24. (canceled)
